(19) United States
(12) Patent Application Publication
(10) Pub. No.: US 2010/0135970 A1
(43) Pub. Date: Jun. 3, 2010

(54) Methods for Reprogramming Adult Somatic Cells and Uses Thereof

(75) Inventors: Raj Kishore, Chicago, IL (US); Douglas W. Losordo, Chicago, IL (US)

Correspondence Address: EDWARDS ANGELL PALMER & DODGE LLP, P.O. BOX 55874, BOSTON, MA, 02205, US

(73) Assignee: Caritas St. Elizabeth Medical Center of Boston, In, Boston, MA (US)

(21) Appl. No.: 12/447,091

(22) PCT Filed: Oct. 26, 2007
(86) PCT No.: PCT/US07/22716

§ 371 Date: Jan. 6, 2010

Related U.S. Application Data
(60) Provisional application No. 60/854,946, filed on Oct. 27, 2006.
(60) Provisional application No. 60/922,221, filed on Apr. 6, 2007.

(51) Int. Cl. A61K 35/12; C12N 5/00; C12N 5/071; C12N 5/079; A61P 9/00

(52) U.S. Cl. 424/93.21; 435/375; 435/366; 435/371; 435/368

(57) ABSTRACT

As described below, the present invention features methods for reprogramming somatic cells and related therapeutic compositions and methods.

### CROSS-REFERENCE TO RELATED APPLICATION

This application claims the benefit of the following U.S. Provisional Application Nos.: 60/922,221, filed Apr. 6, 2007, and 60/854,946, filed Oct. 27, 2006, the contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: AA014575, HL63414, 57516, 53354 66957 and 60911.

### BACKGROUND OF THE INVENTION

A number of clinical trials using autologous bone marrow (BM) and peripheral blood derived stem/progenitor cells have been completed or are currently underway for post-infarct myocardial repair. The available evidence demonstrating improvement in myocardial function following transplantation of autologous BM derived stem/progenitor cells both in pre-clinical as well as in available clinical trials, remains a potent force driving discovery and clinical development simultaneously and has provided new hope for subjects with debilitating heart diseases. Certain potential limitations of autologous BM or peripheral blood derived stem/progenitor cells have been identified. Risk factors for coronary artery disease are reported to be associated with a reduced number and impaired functional activity of endothelial progenitor cells in the peripheral blood of patients. Likewise, patients with diabetes showed lower endothelial progenitor cell numbers. Similarly, in diabetic mice endothelial progenitor cell-mediated re-endothelialization was impaired. Heterogeneity of bone marrow derived stem cells; incomplete mechanistic insights into their function, limited plasticity and trans-differentiation potential to various lineages of cells are also the subject of intense debate. Additionally, the stability with which trans-differentiated cells are able to maintain their newly acquired phenotype and the heritability of this phenotype remains to be defined. Better methods for developing and refining additional sources of autologous cells for tissue repair and regeneration are, therefore, required.

### SUMMARY OF THE INVENTION

As described below, the present invention features methods for de-differentiating and reprogramming somatic cells and related therapeutic compositions and methods.

In one aspect, the invention generally provides a method for generating a reprogrammed cell, the method involves contacting a somatic cell (e.g., fibroblast) containing a permeable cell membrane with an embryonic stem cell extract, thereby generating a de-differentiated cell; and culturing the de-differentiated cell in the presence of at least one agent that induces differentiation, thereby generating a reprogrammed cell (e.g., a mammalian cell line or primary cell). In one embodiment, the method further involves providing the cell to a subject for the repair or regeneration of a tissue or organ. In another embodiment, the method increases function of the tissue or organ. In yet another embodiment, the contacting occurs in an ATP regenerating buffer that contains one or more of ATP, creatine phosphate, and creatine kinase. In still another embodiment, the de-differentiated cell expresses an embryonic stem cell marker not expressed in the somatic cell. In yet another embodiment, the embryonic stem cell marker is any one or more of Nanog, SCF, SSEA1, Oct-4, and c-Kit. In yet another embodiment, the de-differentiated cell has reduced levels of DNA methylation relative to an untreated somatic cell. In yet another embodiment, the de-differentiated cell has increased levels of histone acetylation relative to an untreated somatic cell. In yet another embodiment, the agent is any one or more of LIF, BMP-2, retinoic acid, trans-retinoic acid, dexamethasone, insulin, and indomethacin. In yet another embodiment, the cell is cultured in the presence of LIF and BMP-2 to generate a cardiomyocyte. In yet another embodiment, the reprogrammed cell expresses a cardiomyocyte specific gene any one or more of connexin43, Mef2C, Nkx2.5, GATA4, cardiac troponin I, cardiac troponin T, and Tbx5. In yet another embodiment, the reprogrammed cell expresses two, three, four or more of the cardiomyocyte specific genes. In yet another embodiment, the cell is cultured in the presence of fibronectin and 10% fetal bovine serum to generate an endothelial cell. In yet another embodiment, the endothelial cell expresses an endothelial cell marker that is CD31 or Flk-1. In yet another embodiment, the cell is cultured in the presence of all-trans retinoic acid or a derivative thereof to generate a neuronal cell. In yet another embodiment, the neuronal cell expresses a neuronal marker that is any one or more of nestin and β-tubulin. In yet another embodiment, the cell is cultured in the presence of retinoic acid, dexamethasone, insulin, and/or indomethacin to generate an adipocyte. Preferably, the adipocyte is positive for Oil red O or acetylated LDL uptake.

In yet another aspect, the invention features a method for repairing or regenerating a tissue in a subject, the method involves obtaining the reprogrammed cell of a previous aspect and administering the cell to a subject (e.g., a subject having a myocardial infarction, congestive heart failure, stroke, ischemia, peripheral vascular disease, alcoholic liver disease, cirrhosis, Parkinson's disease, Alzheimer's disease, diabetes, cancer, arthritis, wound healing) and similar diseases, where an increase or replacement of in a particular cell type/ tissue or cellular de-differentiation is desirable. In one embodiment, the subject has damage to the tissue or organ, and the administering provides a dose of cells sufficient to increase a biological function of the tissue or organ or to increase the number of cell present in the tissue or organ. In another embodiment, the subject has a disease, disorder, or condition, and wherein the administering provides a dose of cells sufficient to ameliorate or stabilize the disease, disorder, or condition. In yet another embodiment, the method increases the number of cells of the tissue or organ by at least about 5%, 10%, 25%, 50%, 75% or more compared to a corresponding untreated control tissue or organ. In yet another embodiment, the method increases the biological activity of the tissue or organ by at least about 5%, 10%, 25%, 50%, 75% or more compared to a corresponding untreated control tissue or organ. In yet another embodiment, the method increases blood vessel formation in the tissue or organ by at least about 5%, 10%, 25%, 50%, 75% or more compared to a corresponding untreated control tissue or organ. In yet another embodiment, the cell is administered directly to a subject at a site where an increase in cell number is desired. In one embodiment, the site is a site of tissue damage or disease. In yet another embodiment, the site shows an increase in cell death relative to a corresponding control site.

In yet another aspect, the invention provides a method of ameliorating an ischemic condition in a subject in need thereof, the method involves contacting a fibroblast cell containing a permeable cell membrane with an embryonic stem cell extract; culturing the cell in the presence of LIF and BMP-2 to generate an endothelial cell; and administering the endothelial cell of the previous step into a muscle tissue of the subject, thereby ameliorating an ischemic condition.

In yet another aspect, the invention provides a method of ameliorating a cardiovascular condition in a subject in need thereof, the method involves contacting a somatic cell containing a permeable cell membrane with an embryonic stem cell extract; culturing the cell in the presence of LIF and BMP-2, to generate a cardiomyocyte; and injecting the cardiomyocyte of the previous step into a muscle tissue of the subject, thereby ameliorating a cardiovascular condition. In one embodiment, the method increases left ventricular function, reduces fibrosis, or increases capillary density in a cardiac tissue of the subject. In another embodiment, the contacting is carried out in an ATP regenerating buffer. In yet another embodiment, the method further involves expressing a recombinant protein (e.g., activin A, adrenomedullin, acidic FGF, basic fibroblast growth factor, angiogenin, angiopoietin-1, angiopoietin-2, angiopoietin-3, angiopoietin-4, angiostatin, angiotropin, angiotensin-2, bone morphogenic protein 1, 2, or 3, cadherin, collagen, colony stimulating factor (CSF), endothelial cell-derived growth factor, endoglin, endothelin, endostatin, endothelial cell growth inhibitor, endothelial cell-viability maintaining factor, ephrins, erythropoietin, hepatocyte growth factor, human growth hormone, TNF-alpha, TGF-beta, platelet derived endothelial cell growth factor (PD-ECGF), platelet derived endothelial growth factor (PDGF), insulin-like growth factor-1 or -2 (IGF), interleukin (IL)-1 or 8, FGF-5, fibronectin, granulocyte macrophage colony stimulating factor (GM-CSF), heart derived inhibitor of vascular cell proliferation, IFN-gamma, IGF-2, IFN-gamma, integrin receptor, LIF, leiomyoma-derived growth factor, MCP-1, macrophage-derived growth factor, monocyte-derived growth factor, MMP 2, MMP3, MMP9, neuropilin, neurothelin, nitric oxide donors, nitric oxide synthase (NOS), stem cell factor (SCF), VEGF-A, VEGF-B, VEGF-C, VEGF-D, VEGF-E, VEGF, and VEGF164) in the cell. In yet another embodiment, the recombinant protein is a polypeptide that promotes cell proliferation or differentiation. In one embodiment, the recombinant protein is a reporter protein (e.g., GFP, EGFP, BFP, CFP, YFP, and RFP).

In yet another aspect, the invention provides a reprogrammed cell obtained by the method of any previous aspect. In various embodiments, the cell is a differentiated cardiomyocyte, endothelial cell, neuronal cell, adipocyte, or a precursor thereof.

In yet another aspect, the invention provides a tissue containing the reprogrammed cell of any previous aspect.

In yet another aspect, the invention provides a pharmaceutical composition comprising an effective amount of a cell of any previous aspect in a pharmaceutically acceptable excipient for administration to a subject in need thereof.

In yet another aspect, the invention provides a kit for tissue repair or regeneration comprising a reprogrammed cell obtained by the method of any previous aspect and instructions for use of the cell in methods of tissue repair or regeneration.

In various embodiments of any previous aspect, the subject has damage to the tissue or organ, and the administering provides a dose of cells sufficient to increase a biological function of the tissue or organ. In still other embodiments of the previous aspects, the subject has a disease, disorder, or condition, and wherein the administering provides a dose of cells sufficient to ameliorate or stabilize the disease, disorder, or condition. the method increases the number of cells of the tissue or organ by at least about 5%, 10%, 25%, 50%, 75% or more compared to a corresponding untreated control tissue or organ. In still other embodiments of the previous aspects, the method increases the biological activity of the tissue or organ by at least about 5%, 10%, 25%, 50%, 75% or more compared to a corresponding untreated control tissue or organ. In still other embodiments of the previous aspects, the method increases blood vessel formation in the tissue or organ by at least about 5%, 10%, 25%, 50%, 75% or more compared to a corresponding untreated control tissue or organ. In still other embodiments of the previous aspects, the cell is administered directly to a subject at a site where an increase in cell number is desired. In still other embodiments of the previous aspects, the site is a site of tissue damage or disease. In still other embodiments of the previous aspects, the site shows an increase in cell death relative to a corresponding control site. In still other embodiments of the previous aspects, the subject has a disease that is any one or more of myocardial infarction, congestive heart failure, stroke, ischemia, peripheral vascular disease, alcoholic liver disease, cirrhosis, Parkinson's disease, Alzheimer's disease, diabetes, cancer, arthritis, and wound healing. In still other embodiments of the previous aspects, the method ameliorates ischemic damage. In still other embodiments of the previous aspects, the method reduces apoptosis, increases cell proliferation, increases function, or increases perfusion of muscle tissue (e.g., cardiac tissue or skeletal muscle tissue). In still other embodiments, the method repairs post-infarct ischemic damage in a cardiac tissue. In still other embodiments, the method repairs hind limb ischemia in a skeletal muscle tissue. In still other embodiments, the cell is any of the following: a cardiomyocyte that expresses a cardiomyocyte marker that is any one or more of connexin43, Mef2C, Nkx2.5, GATA4, cardiac troponin I, cardiac troponin T, and Tbx5; an endothelial cell that expresses an endothelial marker that is CD31 or Flk-1; a neuronal cell that expresses a neuronal marker that is nestin or β-tubulin; or an adipocyte cell that is positive for Oil red O.

Other features and advantages of the invention will be apparent from the detailed description, and from the claims.

### Definitions

“Agents” refer to cellular (e.g., biologic) and pharmaceutical factors, preferably growth factors, cytokines, hormones or small molecules, or to genetically-encoded products that modulate cell function (e.g., induce lineage commitment, increase expansion, inhibit or promote cell growth and survival). For example, “expansion agents” are agents that increase proliferation and/or survival of cells of the invention. “Differentiation agents” are agents that induce uncommitted cells to differentiate into committed cell lineages.

By “altered” is meant an increase or decrease. An increase is any positive change, e.g., by at least about 5%, 10%, or 20%; preferably by about 25%, 50%, 75%, or even by 100%, 200%, 300% or more. A decrease is a negative change, e.g., a decrease by about 5%, 10%, or 20%; preferably by about 25%, 50%, 75%; or even an increase by 100%, 200%, 300% or more.

By “angiogenesis” is meant the growth of new blood vessels originating from existing blood vessels. Angiogenesis can be assayed by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area).

By “cell membrane” is meant any membrane that envelops a cell or cellular organelle (e.g., cell nucleus, mitochondria).

By “marker” is meant a gene, polypeptide, modification thereof, or biological function that is characteristic of a particular cell type or cellular phenotype. For example, the expression of embryonic stem cell markers (e.g., Nanog, stem cell factor (SCF), SSEA1, Oct-4, c-Kit, increase in acetylation, decrease in methylation) may be used to characterize a cell as having an embryonic stem cell phenotype. Similarly, the expression of cardiomyocyte specific markers (e.g., cardiotroponin I, Mef2c, connexin43, Nkx2.5, sarcomeric actinin, cariotroponin T and TBX5 may be used to identify a cell as a cardiomyocyte; the expression of endothelial cell specific markers (e.g., CD31) may be used to identify a cell as an endothelial cell; the expression of a muscle specific marker (e.g., desmin) is indicative of muscle cell differentiation; neuronal markers (e.g., nestin and β-tubulin-III) may be used to identify a neuronal cell; adipocyte markers (e.g., Oil-Red-O staining or acetylated LDL uptake) may be used to identify an adipocyte.

The terms “comprises”, “comprising”, and are intended to have the broad meaning ascribed to them in U.S. Patent Law and can mean “includes”, “including” and the like.

By “deficiency of a particular cell-type” is meant fewer of a specific set of cells than are normally present in a tissue or organ not having a deficiency. For example, a deficiency is a 5%, 10%, 15%, 20%, 30%, 40%, 50%, 60%, 70%, 80%, 90%, or even 100% deficit in the number of cells of a particular cell-type (e.g., adipocytes, endothelial cells, endothelial precursor cells, fibroblasts, cardiomyocytes, neurons) relative to the number of cells present in a naturally-occurring, corresponding tissue or organ. Methods for assaying cell-number are standard in the art, and are described in (Bonifacino et al., Current Protocols in Cell Biology, Loose-leaf, John Wiley and Sons, Inc., San Francisco, Calif., 1999; Robinson et al., Current Protocols in Cytometry Loose-leaf, John Wiley and Sons, Inc., San Francisco, Calif., October 1997).

“Derived from” as used herein refers to the process of obtaining a cell from a subject, embryo, biological sample, or cell culture.

“Differentiation” refers to the developmental process of lineage commitment. Differentiation can be assayed by measuring an increase in one or more cell specific markers relative to their expression in a corresponding undifferentiated control cell. A “lineage” refers to a pathway of cellular development, in which precursor or “progenitor” cells undergo progressive physiological changes to become a specified cell type having a characteristic function (e.g., nerve cell, muscle cell or endothelial cell). Differentiation occurs in stages, whereby cells gradually become more specified until they reach full maturity, which is also referred to as “terminal differentiation.” A “terminally differentiated cell” is a cell that has committed to a specific lineage, and has reached the end stage of differentiation (i.e., a cell that has fully matured).

A “de-differentiated cell” is a cell in which the process of differentiation has been, at least to some degree, reversed. De-differentiation can be assayed, for example, by identifying a reduction in the expression of one or more cell specific markers relative to their expression in a corresponding control cell. Alternatively, de-differentiation can be assayed by measuring an increase in one or more markers typically expressed in an embryonic stem cell, a pluripotent or multi-potent cell type, or expressed at an earlier stage of development.

“Engraft” refers to the process of cellular contact and incorporation into a tissue of interest (e.g., muscle tissue) in vivo.

By “stem cell extract” is meant an extract derived at least in part from a stem cell by any chemical or physical process.

The term “isolated” as used herein refers to a cell in a non-naturally occurring state (e.g., isolated from the body or a biological sample).

By “mammal” is meant any warm-blooded animal including but not limited to a human, cow, horse, pig, sheep, goat, bird, mouse, rat, dog, cat, monkey, baboon, or the like. Preferably, the mammal is a human.

By “organ” is meant a collection of cells that perform a biological function. In one embodiment, an organ includes, but is not limited to, bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. The biological function of an organ can be assayed using standard methods known to the skilled artisan.

The term “obtaining” as in “obtaining the agent” is intended to include purchasing, synthesizing or otherwise acquiring the agent (or indicated substance or material).

By “perfused” is meant filled with flowing blood.

By “permeable” is meant allowing the movement of peptides, polypeptides, polynucleotides, and/or small compounds. A permeable cell membrane, for example, provides for the translocation of peptides, polypeptides, polynucleotides, and/or small compounds from one side of a cell membrane to another.

By “positioned for expression” is meant that a polynucleotide (e.g., a DNA molecule) is positioned adjacent to a DNA sequence which directs transcription and, for proteins, translation of the sequence (i.e., facilitates the production of, for example, a recombinant polypeptide of the invention, or an RNA molecule).

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” or “control” is meant a standard condition. For example, an untreated cell, tissue, or organ that is used as a reference.

By “regenerate” is meant capable of contributing at least one cell to the repair or de novo construction of a tissue or organ.

By “repair” is meant to ameliorate damage or disease in a tissue or organ.

By “reprogram” is meant the re-differentiation of a de-differentiated cell.

By “reprogrammed cell” is meant a somatic cell that has undergone de-differentiation and is subsequently induced to re-differentiate. The reprogrammed cell typically expresses a cell specific marker (or set of markers), morphology, and/or biological function that was not characteristic of the cell (or a progenitor thereof) prior to de-differentiation or re-differentiation.

A “somatic” cell refers to a cell that is obtained from a tissue of a subject. Such subjects are at a post-natal stage of development (e.g., adult, infant, child). In contrast, an “embryonic cell” or “embryonic stem cell” is derived from an embryo at a pre-natal stage of development.

The term “subject” as used herein refers to a vertebrate, preferably a mammal (e.g., dog, cat, rodent, horse, bovine, rabbit, goat, or human).

By “tissue” is meant a collection of cells having a similar morphology and function.

By “transformed cell” is meant a cell into which (or into an ancestor of which) has been introduced, by means of recombinant DNA techniques, a polynucleotide molecule encoding (as used herein) a polypeptide of the invention.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “vasculogenesis” is meant the development of new blood vessels originating from stem cells, angioblasts, or other precursor cells.

### BRIEF DESCRIPTION OF THE DRAWINGS

FIGS. 1A-1C show phenotypic changes in 3T3/D3 cells following D3-extract treatment. NIH3T3 fibroblasts were reversibly permeabilized with streptolysin O (SLO) and exposed to D3-mouse embryonic stem cell (mESC) whole cell extracts or to control self (NIH3T3) extract. Cells were cultured in DMEM supplemented with Leukemia Inhibitory Factor (LIF) (10 ng/ml) and monitored daily for morphological changes. FIG. 1A shows four representative phase contrast images of self-extract treated 3T3 on day 10 and D3-extract treated 3T3 on days 3 (d3), 5 (d5) and 10 (d10). Cells were cultured on 4 well slides and expression of c-Kit (FIG. 1B) and Stage Specific Embryonic Antigen 1 (SSEA1) (FIG. 1C) was determined by immuno-fluorescence staining. Six representative photomicrographs obtained using fluorescence microscopy are shown. DAPI was used as a marker for nuclei.

FIGS. 2A-2C show the de-differentiation of mES cell extract treated NIH3T3 cells. FIG. 2A is a graph quantitating mRNA expression by real-time RT-PCR for indicated stem cell markers (e.g., Nanog, stem cell factor (SCF), SSEA1, Oct-4, and c-Kit). Total RNA was extracted from 3T3/D3 four weeks after initiation of treatment. Data is plotted as fold mRNA expression compared to the mRNA levels in self-extract treated 3T3 cells averaged from 3 similar experiments. FIG. 2B presents six photomicrographs showing Oct4 expression determined by immuno-fluorescence staining of cells cultured on 4 well slides. Representative staining is shown. FIG. 2C presents four photomicrographs showing the loss of somatic cell marker, lamin A/C in 3T3/D3 cells as analyzed by immuno-fluorescence cyto-chemistry.

FIGS. 3A-3D show that D3 extract treatment induced epigenetic changes in 3T3/D3 cells. FIG. 3A is a schematic diagram of the Oct4 promoter with the position of CpG indicated. Genomic DNA from indicated cells was digested with EcoRI and treated with sodium meta-bisulphite. The Oct4 promoter was amplified from modified DNA using specific primers by PCR and PCR products were sequenced for the evidence of cytosine conversion to thymine at unmethylated CpG. Filled circles represent methylated CpG and open circles represent unmethylated CpG in the Oct4 promoter. FIG. 3B is a photograph showing a DNA fragment. The Oct4 promoter fragment was amplified from bisulphate treated genomic DNA of indicated cells and was subjected to digestion with HypCH4IV restriction enzyme that specifically cleaved methylated CpG. Post-digestion DNA was resolved on 2% ethidium bromide stained gels and photographed. FIG. 3C is a graph that quantitates histone H3 (AcH3) and H4 acetylation (AcH4) of the Oct4 promoter analyzed by Chromatin Immunoprecipitation (ChIP) relative to control (IgG). FIG. 3D is a graph quantitating the dimethylation status of lysine 9 of histone H3 (diMeK9H3) (3D). Gels from 3 separate experiments were quantified by NIH image analysis and average values were plotted against levels observed in D3 cells (arbitrarily given a numerical value of 1).

FIGS. 4A-4F show global gene expression analysis for re-programmed 3T3/D3 cells. FIG. 4A provides a heatmap of z-scored values for 3286 genes showing significant differences (p<0.001 and absolute log fold change of >1) between 3T3 and 3T3/D3 cells and the expression level of same genes in D3 cells. FIG. 4B shows that genes expressed differentially in 3T3 and 3T3/D3 cells (3286) were grouped in 20 functional categories according to EASE program. FIG. 4C shows a heatmap of the top 500 up-regulated (dark grey) and top 500 down-regulated genes (z-scored values) in 3T3/D3 cells compared to 3T3 cells and relative expression of same genes in D3 cells (* genes up-regulated exclusively in D3 and 3T3/D3; ** top 500 up-regulated genes in 3T3/D3; *** top 500 down-regulated genes in 3T3/D3). FIGS. 4D and 4E provide lists of genes upregulated and downregulated genes, respectively. FIG. 4F provides a list of genes showing significant up-regulation exclusively in D3 and 3T3/D3 cells as compared to 3T3 cells.

FIG. 5A-5D show cardiomyocyte and endothelial cell differentiation of 3T3/D3 cells. Representative phase contrast images of 3T3/D3 cells cultured for 7 days under culture conditions conducive to cardiomyocyte (FIG. 5A, right panel) and endothelial cell (FIG. 5C, right panel) differentiation conditions. FIGS. 5B and 5D are graphs showing fold increase in mRNA expression of cardiomyocyte specific markers cardiotroponin I, Mef2c, connexin43, Nkx2.5, sarcomeric actinin, cariotroponin T and TBX5 (FIG. 5B) and endothelial cell specific markers (FIG. 5D) in 3T3/D3 cells when cultured under cardiomyocyte cell (CMC) and/or endothelial cell (EC) differentiation conditions, in vitro (see materials and methods).

FIGS. 6A and 6B show that de-differentiated 3T3/D3 cells differentiate into cells representative of all 3 germ layers. FIG. 6A shows that under culture conditions conducive to cell specific differentiation, 3T3/D3 cells show protein expression of neuronal (a), cardiomyocyte (b), endothelial cell, merged image of (c) and adipocyte (d) specific markers. FIG. 6B shows that 3T3/D3 subcutaneously injected in SCID mice form teratomas exhibiting differentiation into cell lineages representative of all 3 germ layers.

FIGS. 7A and 7B show that 3T3/D3 cells form teratomas in SCID mice. FIG. 7A shows photographs of mice forming teratomas when injected with D3 and 3T3/D3 cells. FIG. 7B shows the kinetics of tumor growth in mice injected with 3T3, D3 and 3T3/D3 cells. 3T3 cells did not form teratomas until 7 weeks post-injection of cells. D3 cell injected mice revealed tumor growth of ˜3 cm by 3 weeks and were sacrificed at that time.

FIGS. 8A-8D are graphs showing that transplantation of 3T3/D3 cells improves left ventricular function and histological repair in a mouse model of acute myocardial infarction. Transplantation of 3T3/D3 cells significantly improved left ventricular end-diastolic areas (LVEDA) (FIG. 8A), and left ventricular fractional shortening (FIG. 8B) as compared to mice treated with control 3T3 cells and/or saline. FIG. 8C shows the quantification of % fibrosis area in 3 groups of mice. FIG. 8D shows the quantification of capillary density. Mice were perfused with FITC-BS1 lectin and fluorescently labeled capillaries were counted in 6 randomly selected tissue sections at the border zone from each animal.

FIG. 9A-9D shows that 3T3/D3 cells trans-differentiate into cardiomyocytes and endothelial cells, in vivo. FIGS. 9A and 9B are photomicrographs of representative merged figures showing co-localization of GFP+ (green) transplanted control 3T3 cells (left panel) and 3T3/D3 cells (right panel) and CMC specific marker, sarcomeric actinin+ (red) cells. Double positive cells are identified by yellow fluorescence in the merged images (white arrowheads). FIGS. 9C and 9D are representative merged images showing co-localization of GFP+ (green) transplanted 3T3 control (left panel) and 3T3/D3 (right panel) and endothelial cell (EC) specific marker, CD31+ (red) cells. Double positive cells are identified by yellow fluorescence (white arrowheads) in the merged images.

FIG. 10 provides six photomicrographs showing that 3T3/D3 cells incorporate into the vasculature and transdifferentiate into cardiomyocytes, in vivo. Four weeks after AMI and GFP-tagged 3T3 or 3T3/D3 cell transplantation, a subset of mice was perfused with fluorescently labeled BS-1 lectin (red). Myocardium was harvested, fixed with 4% PFA and sectioned. Tissue sections were then analyzed by laser confocal microscopy to visualize co-localization of GFP+ wells with BS-1 lectin stained vasculature. As shown in FIG. 10, GFP+ 3T3/D3 cells (green) co-localized with BS-1 lectin stained vessel (red), giving an yellow fluorescence, while no GFP+ 3T3 cells incorporated into the vasculature (upper panels). Transplanted GFP+ 3T3 or 3T3/D3 cells were also additional cardiomyocyte specific markers (connexin43 and Cardiotroponin I; middle and lower panels) to assess their differentiation to CMC lineage in vivo.

FIGS. 11A and 11B show that 3T3/D3 cell transplantation decreases post-infarct myocardial apoptosis. The number of apoptotic and proliferating myocardial cells in the infracted myocardium 28 days was determined following AMI and cell transplantation. The number of apoptotic cells, as evident from TUNEL+ cells, was significantly higher in myocardial sections of mice treated with control 3T3 as compared to those treated with 3T3/D3 cells (FIG. 11A; 18±2.3 TUNEL+ cells/high visual field in control 3T3 group vs. 5±1.2 TUNEL+ cells/high visual field in 3T3/D3 cell group; p<0.01). A higher number of proliferating cells, (nuclei stained positive for Ki67) was also observed in the myocardial sections from 3T3/D3 treated mice as compared to control 3T3 treated mice (FIG. 11B, p<0.05).

FIGS. 12A-12D show that transplantation of 3T3/D3 cells into a surgically induced mouse hind limb ischemia model improved functional blood flow recovery and neo-vascularization. To ascertain the functional efficacy of re-programmed D3-extract treated 3T3 cells in a physiologically relevant model of tissue repair, studies were conducted in a well-established mouse hind limb ischemia model described in (R. Kishore et al., J Clin Invest. 115, 1785 (2005). Laser Doppler Perfusion Imaging (LDPI), just after the surgery, confirmed establishment of ischemia (complete loss of perfusion in the operated limb; dark shading FIG. 11A left panels). Immediately following the surgery (post-op d0), mice were assigned to two groups and 3T3/D3 or 3T3 cells (2×10⁵), labeled with DiI for tracing purposes, were injected into the ischemic muscles at 3 different sites. Physiological blood flow recovery was assessed by LDPI on day 7 post-surgery (post-op d7), in both groups of mice. As shown in representative perfusion images in FIG. 12A (right panel) and quantified as the ratio of blood flow in ischemic to non-ischemic limb, in FIG. 12B, mice transplanted with 3T3/D3 cells, displayed significantly improved perfusion on day 7 compared to mice treated with 3T3 control cells (p<0.01). This data suggest that transplantation of 3T3/D3 cells into surgically induced mouse hind limb ischemia model improved functional blood flow recovery. To substantiate the physiological blood flow recovery with the anatomical evidence, the number of capillaries in at least 6 randomly selected tissue sections obtained from both group of mice was first determined. Capillaries were identified as fluorescent structures (green), stained with in vivo perfuse FITC-BS-1 lectin. The transplanted cells were traced by red fluorescence (DiI). The number of capillaries/per high visual field in different sections was quantified and averaged. As shown in FIG. 12C, the capillary density was significantly higher in mice that received 3T3/D3 cells compared to those that received control 3T3 cells. Furthermore, 3T3/D3 cells displayed a better proliferative capacity in the ischemic hind limbs than the control 3T3 cells. Immunofluorescence staining for BrdU+DiI double positive cells (indicating proliferation of transplanted cells) revealed a significantly higher number of in vivo proliferating 3T3/D3 cells as compared to control 3T3 cells (FIG. 12D).

FIG. 13 shows the in vivo differentiation of D3-extract treated cells to endothelial and muscle cells was corroborated by co-staining of DiI-labeled cells with specific markers of endothelial and muscle cells. Tissue sections from ischemic hind limbs were stained with mouse FITC-labeled anti-CD31 and anti-desmin antibodies. Fluorescent microscopy was conducted to visualize CD31+ (green) and DiI+ (red) cells and desmin+ (green) and DiI+ (red) cells to determine EC and muscle differentiation, respectively, of transplanted cells and images in the same visual field were merged to generate composite image. As shown in FIG. 13 (panels a, b), many CD31+DiI double positive cells (indicated by arrows) were observed in the ischemic tissue of mice treated with 3T3/D3 cells (panel a) compared to those treated with control 3T3 cells (panel b). Similarly, a large number of 3T3/D3 cells co-expressed muscle marker, desmin, in the ischemic hind limbs while very few desmin+DiI double positive cells in control 3T3 treated mice were observed (FIG. 13, panels c and d). Taken together, these data indicate that re-programmed somatic cells are capable of multi-lineage differentiation in vivo and participate in tissue repair and regeneration.

### DETAILED DESCRIPTION OF THE INVENTION

The invention features compositions and methods that are useful for reprogramming somatic cells and related therapeutic compositions and methods.

Reverse lineage-commitment of adult somatic cells provides an attractive, oocyte-independent source for the generation of pluripotent, autologous stem cells for regenerative medicine. As reported in more detail below, when reversibly permeabilized NIH3T3 cells were exposed to mouse embryonic cell (ESC) extracts, the cells underwent dedifferentiation followed by stimulus-induced re-differentiation or reprogramming into multiple lineage cell types. Genome-wide expression profiling revealed significant differences between NIH3T3 control cells and ESC extract treated NIH3T3 cells, including the up-regulation of ESC specific transcripts. ESC extracts induced CpG de-methylation of Oct4 promoter and hyper-acetylation of histone 3 and 4 as well as decreased dimethylation of histone 3. In a mouse model of acute myocardial infarction (AMI), transplantation of reprogrammed NIH3T3 cells significantly improved post-MI left ventricular function, decreased fibrosis, enhanced capillary density and the transplanted cells trans-differentiated into cardiomyocytes and endothelial cells. Moreover, when injected into SCID mice reprogrammed cells formed teratomas. Taken together these data indicate the oocyte-independent generation of functional autologous stem like cells from terminally differentiated somatic cells.

Somatic cells can be isolated from a number of sources, for example, from biopsies or autopsies using standard methods. The isolated cells are preferably autologous cells obtained by biopsy from the subject. The cells from biopsy can be expanded in culture. Cells from relatives or other donors of the same species can also be used with appropriate immunosuppression. Methods for the isolation and culture of cells are discussed in Fauza et al. (J. Ped. Surg. 33, 7-12, 1998)

Cells are isolated using techniques known to those skilled in the art. For example, a tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with digestive enzymes (e.g., tlypsin, chymotrypsin, collagenase, elastase, hyaluronidase, DNase, pronase, and dispase). Mechanical disruption can be accomplished by scraping the surface of the organ, the use of grinders, blenders, sieves, homogenizers, pressure cells, or sonicators. For a review of tissue disruption techniques, see Freshney, (Culture of Animal Cells. A Manual of Basic Technique, 2d Ed., A. R. Liss, Inc., New York, Ch. 9, pp. 107-126, 1987). While the Examples provided below describe embodiments where fibroblast cells are de-differentiated then reprogrammed, the invention is not so limited. One skilled in the art will readily appreciate that the invention may be employed for the reprogramming of virtually any somatic cell of interest. Moreover, a reprogrammed cell can generate any of a variety of mammalian primary cells or cell lines, with cell types including, without limitation, adipocytes, preadipocytes, urothelial cells, mesenchymal cells, especially smooth or skeletal muscle cells, myocytes (muscle stem cells), mesenchymal precursor cells, cardiac myocytes, fibroblasts, chondrocytes, fibromyoblasts, ectodermal cells ductile cells, and skin cells, hepotocytes, islet cells, cells present in the intestine, parenchymal cells, other cells forming bone or cartilage (e.g., osteoblasts), and neurons.

Once a tissue has been reduced to a suspension of individual cells, the suspension can be fractionated into subpopulations. This may be accomplished using standard techniques (e.g., cloning and positive selection of specific cell types or negative selection, i.e., the destruction of unwanted cells). Selection techniques include separation based upon differential cell agglutination in a mixed cell population, freeze-thaw procedures, differential adherence properties of the cells in the mixed population, filtration, conventional and zonal centrifugation, unit gravity separation, countercurrent distribution, electrophoresis and fluorescence-activated cell sorting. For a review of clonal selection and cell separation techniques, see Freshney, Culture of Animal Cells. A Manual of Basic Techniques, 2d Ed., A. R. Liss, Inc., New York, Ch. 11 and 12, pp. 137-168, 1987). The use of allogenic cells, and more preferably autologous cells, is preferred to prevent tissue rejection. However, if an immunological response does occur in the subject after implantation of the reprogrammed cell, the subject may be treated with immunosuppressive agents, such as cyclosporin or FK506, to reduce the likelihood of rejection.

### Therapeutic and Prophylactic Applications

The present invention provides a ready supply of stem-like cells that could be generated from individual subjects, obviating ethical concerns and also circumventing issues regarding immune rejection, the “stem” cells generated from each individual would be genetically identical to the donor/recipient. The invention also provides methods of using these “stem” cells to repair or regenerate diseased or damaged tissues and organs. In particular embodiments, the invention may be used to increase the number of cells in a tissue or organ having a deficiency in cell number or an excess in cell death. Cells of the invention are administered (e.g., directly or indirectly) to a damaged or diseased tissue or organ where they engraft and increase tissue or organ function. In one embodiment, transplanted cells of the invention function in blood vessel formation to increase perfusion in a damaged tissue or organ, improving organ biological function, reducing apoptosis, and/or reducing fibrosis. These methods may stabilize a damaged tissue or organ in a subject; or the methods may repair or regenerate a damaged or diseased tissue or organ. Methods for repairing damaged tissue or organs may be carried out either in vitro, in vivo, or ex vivo.

Thus, the invention provides methods of treating a disease and/or disorders or symptoms thereof characterized by a deficiency in cell number or excess cell death which comprise administering a therapeutically effective amount of a cellular composition described herein to a subject (e.g., a mammal, such as a human). In one embodiment, the invention provides a method of treating a subject suffering from or susceptible to a disease characterized by a deficiency in cell number or excess cell death (e.g., heart attack, congestive, heart failure, stroke, Parkinson's disease, Alzheimer's disease, diabetes, cancer, arthritis) or disorder or symptom thereof. In particular embodiments, autologous cells could be generated for use in any tissue repair or regeneration indication, including but not limited to, myocardial infarction, congestive heart failure, stroke, ischemia, peripheral vascular diseases, alcoholic liver disease, cirrhosis, Parkinson's disease, Alzheimer's disease, diabetes, cancer, arthritis, wound healing and similar diseases where an increase or replacement of in a particular cell type/ tissue or cellular de-differentiation is desirable. The method includes the step of administering to the mammal a therapeutic amount of a cellular composition herein sufficient to treat the disease or disorder or symptom thereof, under conditions such that the disease or disorder is treated.

The methods herein include administering to the subject (including a subject identified as in need of such treatment) an effective amount of a composition described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the composition herein, such as a composition comprising de-differentiated or reprogrammed cells herein to a subject (e.g., animal, human) in need thereof; including a mammal, particularly a human. Such treatment will be suitably administered to subjects, particularly humans, suffering from, having, susceptible to, or at risk for a disease characterized by a deficiency in cell number or an increase in cell death, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which a deficiency in cell number or an excess in cell death may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a deficiency in cell number or an excess in cell death, in which the subject has been administered a therapeutic amount of a composition herein sufficient to treat the disease or symptoms thereof. The level of Marker determined in the method can be compared to known levels of Marker in either healthy normal controls or in other afflicted subjects to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Methods for Evaluating Therapeutic Efficacy

Methods of the invention are useful for treating or stabilizing in a subject (e.g., a human or mammal) a condition, disease, or disorder affecting a tissue or organ. Therapeutic efficacy is optionally assayed by measuring, for example, the biological function of the treated organ (e.g., bladder, bone, brain, breast, cartilage, esophagus, fallopian tube, heart, pancreas, intestines, gallbladder, kidney, liver, lung, nervous tissue, ovaries, prostate, skeletal muscle, skin, spinal cord, spleen, stomach, testes, thymus, thyroid, trachea, ureter, urethra, urogenital tract, and uterus). Such methods are standard in the art and are described, for example, in the Textbook of Medical Physiology, Tenth edition, (Guyton et al., W.B. Saunders Co., 2000). Preferably, a method of the present invention, increases the biological function of a tissue or organ by at least 5%, 10%, 20%, 40%, 50%, 60%, 70%, 80%, 90%, 100%, 150%, 200%, or even by as much as 300%, 400%, or 500%. In addition, the therapeutic efficacy of the methods of the invention can optionally be assayed by measuring an increase in cell number in the treated or transplanted tissue or organ as compared to a corresponding control tissue or organ (e.g., a tissue or organ that did not receive treatment). Preferably, cell number in a tissue or organ is increased by at least 5%, 10%, 20%, 40%, 60%, 80%, 100%, 150%, or 200% relative to a corresponding tissue or organ. Methods for assaying cell proliferation are known to the skilled artisan and are described in Bonifacino et al., (Current Protocols in Cell Biology Loose-leaf, John Wiley and Sons, Inc., San Francisco, Calif.). Alternatively, the therapeutic efficacy of the methods of the invention is assayed by measuring angiogenesis, blood vessel formation, blood flow, or the function of a blood vessel network in the tissue or organ receiving treatment as compared to a control tissue or organ (e.g., corresponding tissue or organ that did not receive treatment). A method that increases blood vessel formation or perfusion (e.g., by at least 5%, 10%, 20%, 30%, 40%, 50%, 60%, 70%, 80%, 100%, 150%, or 200%, or even by as much as 300%, 400%, or 500%) is considered to be useful in the invention. Methods for evaluating angiogenesis and vasculogenesis are standard in the art and are described herein.

### Administration

Cells of the invention include somatic cells that have been de-differentiated and reprogrammed or re-differentiated to express cell specific markers. Such cells can be provided directly to a tissue or organ of interest (e.g., by direct injection). In one embodiment, cells of the invention are provided to a site where an increase in the number of cells is desired, for example, due to disease, damage, injury, or excess cell death. Alternatively, cells of the invention can be provided indirectly to a tissue or organ of interest, for example, by administration into the circulatory system. If desired, the cells are delivered to a portion of the circulatory system that supplies the tissue or organ to be repaired or regenerated. Advantageously, cells of the invention engraft within the tissue or organ. If desired, expansion and differentiation agents can be provided prior to, during or after administration of the cells to increase, maintain, or enhance production or differentiation of the cells in vivo. Compositions of the invention include pharmaceutical compositions comprising reprogrammed cells or their progenitors and a pharmaceutically acceptable carrier. Administration can be autologous or heterologous. For example, cells obtained from one subject, can be administered to the same subject or a different, compatible subject. Methods for administering cells are known in the art, and include, but are not limited to, catheter administration, systemic injection, localized injection, intravenous injection, intramuscular, intracardiac injection or parenteral administration. When administering a therapeutic composition of the present invention (e.g., a pharmaceutical composition), it will generally be formulated in a unit dosage injectable form (solution, suspension, emulsion).

### Formulations

Cellular compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells (e.g., de-differentiated or reprogrammed cells) utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the de-differentiated cells or reprogrammed cells or their progenitors.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

A method to potentially increase cell survival when introducing the cells into a subject is to incorporate cells or their progeny (e.g., in vivo, ex vivo or in vitro derived cells) of interest into a biopolymer or synthetic polymer. Depending on the subject's condition, the site of injection might prove inhospitable for cell seeding and growth because of scarring or other impediments. Examples of biopolymer include, but are not limited to, cells mixed with fibronectin, fibrin, fibrinogen, thrombin, collagen, and proteoglycans. This could be constructed with or without included expansion or differentiation factors. Additionally, these could be in suspension, but residence time at sites subjected to flow would be nominal. Another alternative is a three-dimensional gel with cells entrapped within the interstices of the cell biopolymer admixture. Again, expansion or differentiation factors could be included with the cells. These could be deployed by injection via various routes described herein.

Exemplary agents that may be delivered together with a reprogrammed or de-differentiated cell of the invention include, but are not limited to, any one or more of activin A, adrenomedullin, acidic FGF, basic fibroblast growth factor, angiogenin, angiopoietin-1, angiopoietin-2, angiopoietin-3, angiopoietin-4, angiostatin, angiotropin, angiotensin-2, bone morphogenic protein 1, 2, or 3, cadherin, collagen, colony stimulating factor (CSF), endothelial cell-derived growth factor, endoglin, endothelin, endostatin, endothelial cell growth inhibitor, endothelial cell-viability maintaining factor, ephrins, erythropoietin, hepatocyte growth factor, human growth hormone, TNF-alpha, TGF-beta, platelet derived endothelial cell growth factor (PD-ECGF), platelet derived endothelial growth factor (PDGF), insulin-like growth factor-1 or -2 (IGF), interleukin (IL)-1 or 8, FGF-5, fibronectin, granulocyte macrophage colony stimulating factor (GM-CSF), heart derived inhibitor of vascular cell proliferation, IFN-gamma, IFN-gamma, integrin receptor, LIF, leiomyoma-derived growth factor, MCP-1, macrophage-derived growth factor, monocyte-derived growth factor, MMP 2, MMP3, MMP9, neuropilin, neurothelin, nitric oxide donors, nitric oxide synthase (NOS), stem cell factor (SCF), VEGF-A, VEGF-B, VEGF-C, VEGF-D, VEGF-E, VEGF, and VEGF164. Other agents that may be delivered together with a cell of the invention include one or more of LIF, BMP-2, retinoic acid, trans-retinoic acid, dexamethasone, insulin, indomethacin, fibronectin and/or 10% fetal bovine serum, or a derivative thereof. Preferably, a cell of the invention is delivered together with a combination of LIF and BMP-2; with fibronectin and 10% fetal bovine serum; with retinoic acid or a derivative thereof together with mitotic inhibitors, such as fluorodeoxyuridine, cytosine arabinosine, and uridine; and dexamethasone, insulin, and/or indomethacin.

Those skilled in the art will recognize that the polymeric components of the compositions should be selected to be chemically inert and will not affect the viability or efficacy of the reprogrammed or de-differentiated cells or their progenitors as described in the present invention. This will present no problem to those skilled in chemical and pharmaceutical principles, or problems can be readily avoided by reference to standard texts or by simple experiments (not involving undue experimentation), from this disclosure and the documents cited herein.

### Dosages

One consideration concerning the therapeutic use of reprogrammed or de-differentiated cells or their progenitors of the invention is the quantity of cells necessary to achieve an optimal effect. In general, doses ranging from 1 to 4×10⁷ cells may be used. However, different scenarios may require optimization of the amount of cells injected into a tissue of interest. Thus, the quantity of cells to be administered will vary for the subject being treated. In a preferred embodiment, between 10⁴ to 10⁸, more preferably 10⁵ to 10⁷, and still more preferably, 1, 2, 3, 4, 5, 6, 7×10⁷ stem cells of the invention can be administered to a human subject.

Fewer cells can be administered directly a tissue where an increase in cell number is desirable. Preferably, between 10² to 10⁶, more preferably 10³ to 10⁵, and still more preferably, 10⁴ reprogrammed or de-differentiated cells or their progenitors can be administered to a human subject. However, the precise determination of what would be considered an effective dose may be based on factors individual to each subject, including their size, age, sex, weight, and condition of the particular subject. As few as 100-1000 cells can be administered for certain desired applications among selected patients. Therefore, dosages can be readily ascertained by those skilled in the art from this disclosure and the knowledge in the art.

Reprogrammed or de-differentiated cells or their progenitors of the invention can comprise a purified population of reprogrammed or de-differentiated cells. As described herein, cells of the invention are identified as de-differentiated or reprogrammed, for example, by the expression of markers, by cellular morphology, or by the ability to form a particular cell type (e.g., ectodermal cell, mesodermal cell, endodermal cell, adipocyte, myocyte, neuron). Those skilled in the art can readily determine the percentage of cells in a population using various well-known methods, such as fluorescence activated cell sorting (FACS). Preferable ranges of purity in populations comprising reprogrammed or de-differentiated cells are about 50 to about 55%, about 55 to about 60%, and about 65 to about 70%. More preferably the purity is about 70 to about 75%, about 75 to about 80%, about 80 to about 85%; and still more preferably the purity is about 85 to about 90%, about 90 to about 95%, and about 95 to about 100%. Purity of reprogrammed or de-differentiated cells or their progenitors can be determined according to the marker profile within a population. Dosages can be readily adjusted by those skilled in the art (e.g., a decrease in purity may require an increase in dosage).

The skilled artisan can readily determine the amount of cells and optional additives, vehicles, and/or carrier in compositions and to be administered in methods of the invention. Typically, any additives (in addition to the active stem cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

If desired, cells of the invention are delivered in combination with (prior to, concurrent with, or following the delivery of) agents that increase survival, increase proliferation, enhance differentiation, and/or promote maintenance of a differentiated cellular phenotype. Expansion agents include growth factors that are known in the art to increase proliferation or survival of stem cells. Such agents are expected to be similarly useful for the expansion of cells of the invention, particularly for the expansion of de-differentiated cells. For example, U.S. Pat. Nos. 5,750,376 and 5,851,832 describe methods for the in vitro culture and proliferation of neural stem cells using transforming growth factor. An active role in the expansion and proliferation of stem cells has also been described for BMPs (Zhu, G. et al, (1999) Dev. Biol. 215: 118-29 and Kawase, E. et al, (2001) Development 131: 1365), LIF (Menard C et al (2005), Lancet. 366:1005-1012) and Wnt proteins (Pazianos, G. et al, (2003) Biotechniques 35: 1240 and Constantinescu, S. (2003) J. Cell Mol. Med. 7: 103). U.S. Pat. Nos. 5,453,357 and 5,851,832 describe proliferative stem cell culture systems that utilize fibroblast growth factors. The contents of each of these references are specifically incorporated herein by reference for their description of expansion agents known in the art.

Agents comprising growth factors are also known in the art to increase mobilization of stem cells from the bone marrow into the peripheral blood. Mobilizing agents include but are not limited to GCSF or GMCSF. An agent that increases mobilization of stem cells into the blood can be provided to augment or supplement other methods of the invention where it would be desirable to increase circulating levels of bone marrow derived stem cells (e.g., to increase engraftment of such cells in an ischemic tissue).

Agents comprising growth factors are known in the art to differentiate stem cells. Such agents are expected to be similarly useful for inducing the re-differentiation or reprogramming of de-differentiated cells. For example, TGF-β can induce differentiation of hematopoietic stem cells (Ruscetti, F. W. et al, (2001) Int. J. Hematol. 74: 18). U.S. Patent Application No. 2002142457 describes methods for differentiation of cardiomyocytes using BMPs. Pera et al describe human embryonic stem cell differentiation using BMP-2 (Pera, M. F. et al, (2004) J. Cell Sci. 117: 1269). U.S. Patent Application No. 20040014210 and U.S. Pat. No. 6,485,972 describe methods of using Wnt proteins to induce differentiation. U.S. Pat. No. 6,586,243 describes differentiation of dendritic cells in the presence of SCF. U.S. Pat. No. 6,395,546 describes methods for generating dopaminergic neurons in vitro from embryonic and adult central nervous system cells using LIF. The contents of each of these references are specifically incorporated herein by reference for their description of differentiation agents known in the art.

In vitro and ex vivo applications of the invention involve the culture of de-differentiated cells or reprogrammed cells or their progenitors with a selected agent to achieve a desired result. Cultures of cells (from the same individual and from different individuals) can be treated with expansion agents prior to, during, or following de-differentiation to increase the number of cells suitable for reprogramming. Similarly, differentiation agents of interest can be used to reprogram a de-differentiated cell, which can then be used for a variety of therapeutic applications (e.g., tissue or organ repair, regeneration, treatment of an ischemic tissue, or treatment of myocardial infarction).

If desired, de-differentiated or reprogrammed cells of the invention are delivered in combination with other factors that promote cell survival, differentiation, or engraftment. Such factors, include but are not limited to nutrients, growth factors, agents that induce differentiation or de-differentiation, products of secretion, immunomodulators, inhibitors of inflammation, regression factors, hormones, or other biologically active compounds. Exemplary agents include, but are not limited to

### Delivery Methods

Compositions of the invention (e.g., cells in a suitable vehicle) can be provided directly to an organ of interest, such as an organ having a deficiency in cell number as a result of injury or disease. Alternatively, compositions can be provided indirectly to the organ of interest, for example, by administration into the circulatory system.

Compositions can be administered to subjects in need thereof by a variety of administration routes. Methods of administration, generally speaking, may be practiced using any mode of administration that is medically acceptable, meaning any mode that produces effective levels of the active compounds without causing clinically unacceptable adverse effects. Such modes of administration include intramuscular, intra-cardiac, oral, rectal, topical, intraocular, buccal, intravaginal, intracisternal, intracerebroventricular, intratracheal, nasal, transdermal, within/on implants, e.g., fibers such as collagen, osmotic pumps, or grafts comprising reprogrammed or de-differentiated cells, etc., or parenteral routes. The term “parenteral” includes subcutaneous, intravenous, intramuscular, intraperitoneal, intragonadal or infusion. A particular method of administration involves coating, embedding or derivatizing fibers, such as collagen fibers, protein polymers, etc. with therapeutic proteins. Other useful approaches are described in Otto, D. et al., J. Neurosci. Res. 22: 83 and in Otto, D. and Unsicker, K. J. Neurosci. 10: 1912.

In one approach, re-differentiated cells derived from cultures of the invention are implanted into a host. The transplantation can be autologous, such that the donor of the cells is the recipient of the transplanted cells; or the transplantation can be heterologous, such that the donor of the cells is not the recipient of the transplanted cells. Once transferred into a host, the re-differentiated cells are engrafted, such that they assume the function and architecture of the native host tissue.

In another approach, de-differentiated cells derived from cultures of the invention are implanted into a host. The transplantation can be autologous, such that the donor of the cells is the recipient of the transplanted cells; or the transplantation can be heterologous, such that the donor of the cells is not the recipient of the transplanted cells. Once transferred into a host, the de-differentiated cells are induced to undergo re-differentiation. The reprogrammed cells are then engrafted, such that they assume the function and architecture of the native host tissue.

De-differentiated cells and reprogrammed cells and the progenitors thereof can be cultured, treated with agents and/or administered in the presence of polymer scaffolds. If desired, agents described herein are incorporated into the polymer scaffold to promote cell survival, proliferation, enhance maintenance of a cellular phenotype. Polymer scaffolds are designed to optimize gas, nutrient, and waste exchange by diffusion. Polymer scaffolds can comprise, for example, a porous, non-woven array of fibers. The polymer scaffold can be shaped to maximize surface area, to allow adequate diffusion of nutrients and growth factors to the cells. Taking these parameters into consideration, one of skill in the art could configure a polymer scaffold having sufficient surface area for the cells to be nourished by diffusion until new blood vessels interdigitate the implanted engineered-tissue using methods known in the art. Polymer scaffolds can comprise a fibrillar structure. The fibers can be round, scalloped, flattened, star-shaped, solitary or entwined with other fibers. Branching fibers can be used, increasing surface area proportionately to volume.

Unless otherwise specified, the term “polymer” includes polymers and monomers that can be polymerized or adhered to form an integral unit. The polymer can be non-biodegradable or biodegradable, typically via hydrolysis or enzymatic cleavage. The term “biodegradable” refers to materials that are bioresorbable and/or degrade and/or break down by mechanical degradation upon interaction with a physiological environment into components that are metabolizable or excretable, over a period of time from minutes to three years, preferably less than one year, while maintaining the requisite structural integrity. As used in reference to polymers, the term “degrade” refers to cleavage of the polymer chain, such that the molecular weight stays approximately constant at the oligomer level and particles of polymer remain following degradation.

Materials suitable for polymer scaffold fabrication include polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon®, nylon silicon, and shape memory materials, such as poly(styrene-block-butadiene), polynorbornene, hydrogels, metallic alloys, and oligo(ε-caprolactone)diol as switching segment/oligo(p-dioxyanone)diol as physical crosslink. Other suitable polymers can be obtained by reference to The Polymer Handbook, 3rd edition (Wiley, N.Y., 1989).

### Kits

De-differentiated or reprogrammed cells of the invention may be supplied along with additional reagents in a kit. The kits can include instructions for the treatment regime or assay, reagents, equipment (test tubes, reaction vessels, needles, syringes, etc.) and standards for calibrating or conducting the treatment or assay. The instructions provided in a kit according to the invention may be directed to suitable operational parameters in the form of a label or a separate insert. Optionally, the kit may further comprise a standard or control information so that the test sample can be compared with the control information standard to determine if whether a consistent result is achieved.

### Screening Assays

The invention provides methods for identifying modulators, i.e., candidate or test compounds or agents (e.g., proteins, peptides, peptidomimetics, peptoids, polynucleotides, small molecules or other agents) which enhance de-differentiation or reprogramming. Agents thus identified can be used to modulate, for example, proliferation, survival, differentiation of cells of the invention, or their progenitors, or maintenance of a cellular phenotype, for example, in a therapeutic protocol.

The test agents of the present invention can be obtained singly or using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. (1994) et al., J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al (1994) Angew. Chem. Int. Ed. Engl. 33:2061; and Gallop et al. (1994) J. Med. Chem. 37:1233.

Libraries of compounds may be presented in solution (e.g., Houghten (1992), Biotechniques 13:412-421), or on beads (Lam (1991), Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Chemical compounds to be used as test agents (i.e., potential inhibitor, antagonist, agonist) can be obtained from commercial sources or can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock (1989) Comprehensive Organic Transformations, VCH Publishers; T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Test agents of the invention can also be peptides (e.g., growth factors, cytokines, receptor ligands). Screening methods of the invention can involve the identification of an agent that increases the proliferation, survival of de-differentiated or reprogrammed cells or the progenitors thereof, or maintenance of a cellular phenotype. Such methods will typically involve contacting a population of the de-differentiated or reprogrammed cells with a test agent in culture and quantitating the number of new de-differentiated or reprogrammed cells produced as a result. Comparison to an untreated control can be concurrently assessed. Where an increase in the number of de-differentiated or reprogrammed cells is detected relative to the control, the test agent is determined to have the desired activity.

In practicing the methods of the invention, it may be desirable to employ a purified population of cells or the progenitors thereof. A purified population of de-differentiated or reprogrammed cells have about 50%, 55%, 60%, 65% or 70% purity. More preferably the purity is about 75%, 80%, or 85%; and still more preferably the purity is about 90%, 95%, 97%, or even 100%.

Increased amounts of de-differentiated or reprogrammed cells or the progenitors thereof can also be detected by an increase in gene expression of genetic markers. For example, de-differentiation is detected by measuring an increase (e.g., 5%, 10%, 25%, 50%, 75% or 100%) in the expression of one or more embryonic stem cell markers, such as Oct4, Nanog, SSEA1, SCF and c-Kit. Further evidence of de-differentiation is shown by a reduction in or the loss of lamin A/C protein expression. Alternatively, de-differentiation is detected by measuring an increase in acetylation, such as increased acetylation of H3 and H4 within the promoter of Oct4, or by measuring a decrease in methylation, for example, by measuring the demethylation of lysine 9 of histone 3. In each of these cases, de-differentiation is measured relative to a control cell. In other embodiments, de-differentiation is assayed by any other method that detects chromatin remodeling leading to the activation of an embryonic stem cell marker, such as Oct4.

Re-differentiation or reprogramming of a de-differentiated cell is detected by assaying increases in expression of cell specific markers that are not typically expressed in the cell from which the reprogrammed cell is derived. An increase in the expression of a cell specific marker may be by about 5%, 10%, 25%, 50%, 75% or 100%. For example, a neuronal cell is detected by assaying for neuronal markers, such as nestin and β-tubulin-III; an adipocyte is detected by assaying for Oil-Red-O staining or acetylated LDL uptake. Cardiomyocytes are detected by assaying for the expression of one or more cardiomyocyte specific markers, such as cardiotroponin I, Mef2c, connexin43, Nkx2.5, sarcomeric actinin, cariotroponin T and TBX5, and sarcomeric actinin. The presence of endothelial cells is detected by assaying the presence of an endothelial cell specific marker, such as CD31+. The level of expression can be measured in a number of ways, including, but not limited to: measuring the mRNA encoded by the markers; measuring the amount of protein encoded by the markers; or measuring the activity of the protein encoded by the markers.

The level of mRNA corresponding to a marker can be determined both by in situ and by in vitro formats. The isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe is sufficient to specifically hybridize under stringent conditions to mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the genetic markers described herein.

The level of mRNA in a sample can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the genetic marker being analyzed.

In other embodiments, de-differentiation or re-differentiation is detected by measuring an alteration in the morphology or biological function of a de-differentiated or re-differentiated cell. An alteration in biological function may be assayed, for example, by measuring an increase in acetylated LDL uptake in a reprogrammed adipocyte. Other methods for assaying cell morphology and function are known in the art and are described in the Examples.

Screening methods of the invention can involve the identification of an agent that increases the differentiation of de-differentiated cells into a cell type of interest. Such methods will typically involve contacting the de-differentiated cells with a test agent in culture and quantitating the number of reprogrammed cells produced as a result. Comparison to an untreated control can be concurrently assessed. Where an increase in the number of reprogrammed cells is detected relative to the control, the test agent is determined to have the desired activity. The test agent can also be assayed using a biological sample (e.g., ischemic tissue); subsequent testing using a population of reprogrammed cells may be conducted to distinguish the functional activity of the agent (e.g., differentiation rather then increase in proliferation or survival) where the result is ambiguous.

### Expression of Recombinant Proteins

In another approach, the de-differentiated cell or reprogrammed cells of the invention may be engineered to express a gene of interest whose expression promotes cell survival, proliferation, differentiation, maintenance of a cellular phenotype, or otherwise enhances the engraftment of the cell. Alternatively, expression of a gene of interest in a cell of the invention may promote the repair or regeneration of a tissue or organ having a deficiency in cell number or excess cell death. Exemplary proteins that may be expressed in a cell of the invention include, but are not limited to, angiopoietin, acidic fibroblast growth factors (aFGF) (GenBank Accession No. NP_{—}149127) and basic FGF (GenBank Accession No. AAA52448), bone morphogenic protein (GenBank Accession No. BAD92827), BMP-2, vascular endothelial growth factor (VEGF) (GenBank Accession No. AAA35789 or NP_{—}001020539), epidermal growth factor (EGF) (GenBank Accession No. NP_{—}001954), transforming growth factor α (TGF-α) (GenBank Accession No. NP_{—}003227) and transforming growth factor β (TFG-β) (GenBank Accession No. 1109243A), platelet-derived endothelial cell growth factor (PD-ECGF) (GenBank Accession No. NP_{—}001944), platelet-derived growth factor (PDGF) (GenBank Accession No. 1109245A), tumor necrosis factor α (TNF-α) (GenBank Accession No. CAA26669), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP_{—}000749) and nitric oxide synthase (NOS) (GenBank Accession No. AAA36365), and fragments or variants thereof. Alternatively, cells of the invention may express a component of the extracellular matrix (ECM). ECM components include structural proteins, such as collagen and elastin; proteins having specialized functions, such as fibrillin, fibronectin, and laminin; and proteoglycans that include long chains of repeating disaccharide units termed of glycosaminoglycans (e.g., hyaluronan, chondroitin sulfate, dermatan sulfate, heparan sulfate, heparin, keratan sulfate, aggrecan).

The gene of interest may be constitutively expressed or its expression may be regulated by an inducible promoter or other control mechanism where conditions necessitate highly controlled regulation or timing of the expression of a protein, enzyme, or other cell product. Such de-differentiated cells or reprogrammed cells, when transplanted into a subject produce high levels of the protein to confer a therapeutic benefit. For example, the cell of the invention, its progenitor or its in vitro-derived progeny, can contain heterologous DNA encoding genes to be expressed, for example, in gene therapy. Insertion of one or more pre-selected DNA sequences can be accomplished by homologous recombination or by viral integration into the host cell genome. The desired gene sequence can also be incorporated into the cell, particularly into its nucleus, using a plasmid expression vector and a nuclear localization sequence. Methods for directing polynucleotides to the nucleus have been described in the art. The genetic material can be introduced using promoters that will allow for the gene of interest to be positively or negatively induced using certain chemicals/drugs, to be eliminated following administration of a given drug/chemical, or can be tagged to allow induction by chemicals, or expression in specific cell compartments.

Calcium phosphate transfection can be used to introduce plasmid DNA containing a target gene or polynucleotide into de-differentiated cells or reprogrammed cells and is a standard method of DNA transfer to those of skill in the art. DEAE-dextran transfection, which is also known to those of skill in the art, may be preferred over calcium phosphate transfection where transient transfection is desired, as it is often more efficient. Since the cells of the present invention are isolated cells, microinjection can be particularly effective for transferring genetic material into the cells. This method is advantageous because it provides delivery of the desired genetic material directly to the nucleus, avoiding both cytoplasmic and lysosomal degradation of the injected polynucleotide. Cells of the present invention can also be genetically modified using electroporation.

Liposomal delivery of DNA or RNA to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPA) can be added. Commercially available reagents for liposomal transfer include Lipofectin (Life Technologies). Lipofectin, for example, is a mixture of the cationic lipid N-[1-(2,3-dioleyloxy)propyl]-N-N-N-trimethyl ammonia chloride and DOPE. Liposomes can carry larger pieces of DNA, can generally protect the polynucleotide from degradation, and can be targeted to specific cells or tissues. Cationic lipid-mediated gene transfer efficiency can be enhanced by incorporating purified viral or cellular envelope components, such as the purified G glycoprotein of the vesicular stomatitis virus envelope (VSV-G). Gene transfer techniques which have been shown effective for delivery of DNA into primary and established mammalian cell lines using lipopolyamine-coated DNA can be used to introduce target DNA into the de-differentiated cells or reprogrammed cells described herein.

Naked plasmid DNA can be injected directly into a tissue comprising cells of the invention (e.g., de-differentiated or reprogrammed cells). This technique has been shown to be effective in transferring plasmid DNA to skeletal muscle tissue, where expression in mouse skeletal muscle has been observed for more than 19 months following a single intramuscular injection. More rapidly dividing cells take up naked plasmid DNA more efficiently. Therefore, it is advantageous to stimulate cell division prior to treatment with plasmid DNA. Microprojectile gene transfer can also be used to transfer genes into cells either in vitro or in vivo. The basic procedure for microprojectile gene transfer was described by J. Wolff in Gene Therapeutics (1994), page 195. Similarly, microparticle injection techniques have been described previously, and methods are known to those of skill in the art. Signal peptides can be also attached to plasmid DNA to direct the DNA to the nucleus for more efficient expression.

Viral vectors are used to genetically alter cells of the present invention and their progeny. Viral vectors are used, as are the physical methods previously described, to deliver one or more target genes, polynucleotides, antisense molecules, or ribozyme sequences, for example, into the cells. Viral vectors and methods for using them to deliver DNA to cells are well known to those of skill in the art. Examples of viral vectors that can be used to genetically alter the cells of the present invention include, but are not limited to, adenoviral vectors, adeno-associated viral vectors, retroviral vectors (including lentiviral vectors), alphaviral vectors (e. g., Sindbis vectors), and herpes virus vectors.

Peptide or protein transfection is another method that can be used to genetically alter de-differentiated cells or reprogrammed cells of the invention and their progeny. Peptides such as Pep-1 (commercially available as Chariot™), as well as other protein transduction domains, can quickly and efficiently transport biologically active proteins, peptides, antibodies, and nucleic acids directly into cells, with an efficiency of about 60% to about 95% (Morris, M. C. et al, (2001) Nat. Biotech. 19: 1173-1176).

The following examples are put forth so as to provide those of ordinary skill in the art with a complete disclosure and description of how to make and use the assay, screening, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

Experimental evidence has revealed nuclear re-programming of terminally differentiated adult mammalian cells leading to their de-differentiation. One example of somatic cell nuclear re-programming comes from reproductive and therapeutic cloning experiments utilizing somatic nuclear transfer (SNT), wherein transplantation of somatic nuclei into enucleated oocyte cytoplasm can extensively reprogram somatic cell nuclei with new patterns of gene expression, new pathways of cell differentiation, generation of embryonic stem cells and birth of cloned animals. Therapeutic cloning, although conceptually attractive, is hampered by the technical challenges, extremely low efficiency, oocyte-dependence, ethico-legal concerns and prohibitive cost associated with the process. Accordingly, alternative strategies for somatic cell re-programming is likely to be important in the development of therapeutics utilizing reprogrammed cells. The present invention provides oocyte-independent systems that involve the exposure of somatic cell nuclei to ESC-derived cell-free factors/proteins to drive somatic cell de-differentiation and nuclear re-programming.

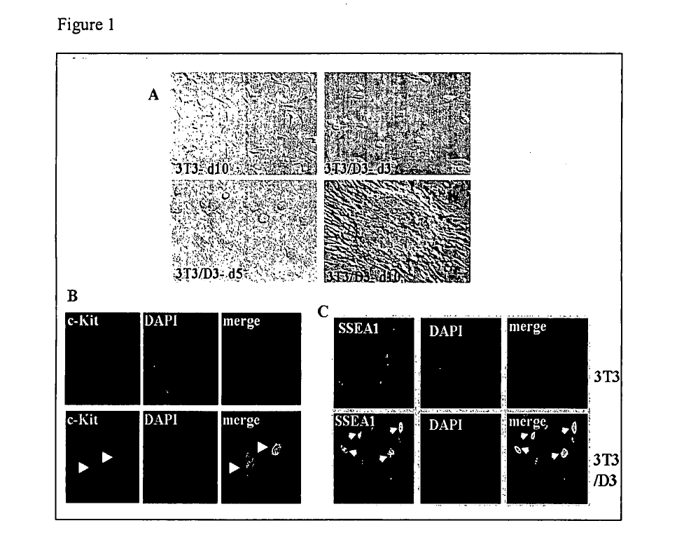

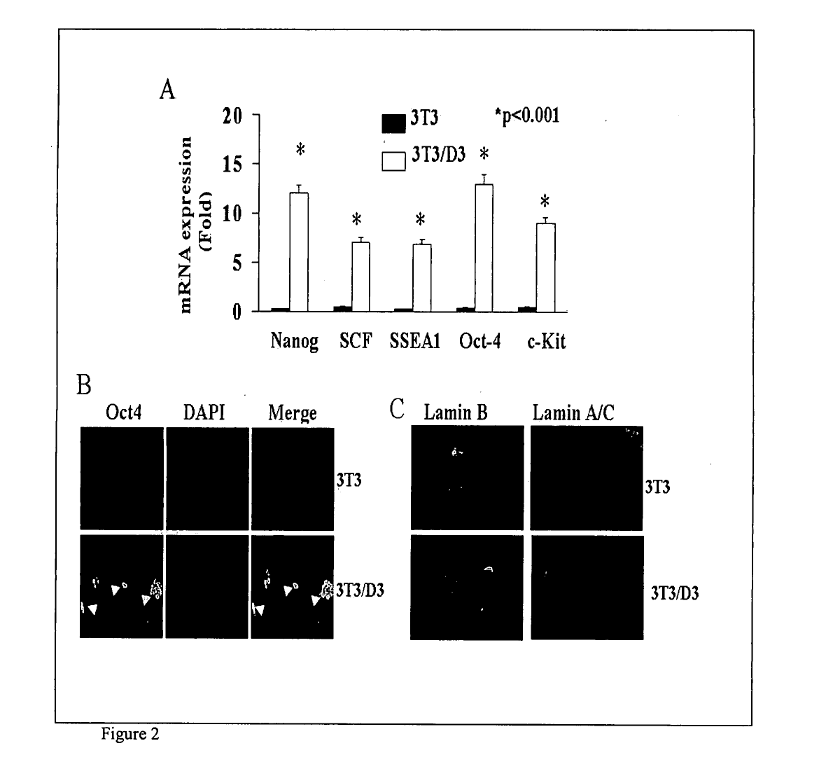

To determine if cell-free extracts from the mouse embryonic stem cell (mESC) line D3, might provide the necessary regulatory proteins to induce de-differentiation followed by stimulus-induced re-differentiation of NIH3T3 murine fibroblasts, reversibly permeabilized NIH3T3 cells were treated with whole cell extracts from NIH3T3 (control) or with mouse embryonic stem cell D3 (ATCC CRL-1934) extracts, made as described below, and the treated cells were cultured in complete DMEM in the presence of LIF (10 ng/ml) for 10 days. As shown in FIG. 1, NIH3T3 cells treated with the self extracts (hereinafter referred to as “3T3 cells”) did not show any morphological changes up to day 10 post-treatment whereas, NIH3T3 cells treated with D3 extracts (hereinafter referred to as “3T3/D3”) showed significant changes in cell morphology on days 3, 5 and 10. On day 10 the 3T3/D3 cells formed colonies resembling typical embryonic stem cell (ESC) morphology. To determine if the altered morphology of 3T3/D3 cells represents the de-differentiation of NIH3T3 cells, the induction of mES markers in 3T3/D3 and 3T3 cells was analyzed up to 4 weeks post-treatment, both at the mRNA level by quantitative real-time PCR and at the protein level by immuno-fluorescent staining. Quantitative mRNA expression of mESC markers, Oct4, Nanog, SSEA1, SCF and c-Kit was significantly higher in 3T3/D3 cells while 3T3 cells did not express measurable mRNA for any of these stem cell markers (FIG. 2A). Enhanced mRNA expression of stem cell specific genes was further corroborated by immuno-fluorescence staining for selected markers Oct4 (FIG. 2B), c-Kit and SSEA1 (FIGS. 1B, and 1C). Further evidence of NIH3T3 de-differentiation is shown by the loss of lamin A/C protein expression in 3T3/D3 cells (FIG. 2C). Lamin A/C is a specific marker of somatic cells. Taken together, these data indicate that cell-free ESC extracts provide the necessary regulatory components required to induce somatic nuclear reprogramming and alter the differentiation status of non-embryonic cells.

DNA methylation of CpG residues leading to the silencing of pluripotent embryonic genes, including that of Oct4, is known as an integral step governing differentiation and development. Since D3-extract exposure leads to the induction of Oct4 mRNA and protein expression in 3T3/D3 cells, studies were performed to determine whether D3-extract treatment lead to demethylation of CpG residues in the promoter of Oct4 gene. The methylation status of each CpG in the Oct4 promoter region was investigated (10 CpG sites) by sodium bisulphite genomic sequencing. The bisulfite-converted genomic DNA (1μg) from D3, 3T3 and 3T3/D3 cells was amplified for Oct4 promoter by PCR. Primers are listed in Table 1 (below).

TABLE 1
Probe/Primer sequences for quantitative PCR
(correct)

| Gene | sequence |
|---|---|
| Nanog | CAGGGCTATCTGGTGAACGCATCTGG |
| GATA-4 | CCCGGGCTGTCATCTCACTATGGG |
| Oct-4 | ATCTCCCCATGTCCGCCCGC |
| c-kit | TGCTGAGCTTCTCCTACCAGGTGGCC |
| CTI | AAGCGGCCCACTCTCCGAAGA |
| Mef2c | TCACCAGACCTTCGCCGGACGA |
| Connexin43 | AGCGATCCTTACCACGCCACCACTG |
| Nkx2.5 | TCCGCGAGCCTACGGTGACCCT |
| Tbx5 | TGTACCGTCACCACCGTGCAGCC |
| CTT | ACTCCGGATTCTGTCTGAGAGGAAAA |
| Flk-1 | ACGCTTGGACAGCATCACCAGCAG |
| CD31 | TTTATGAACCTGCCCTGCTCCCACA |
| SCF c-kit ligand | CAGCCATGGCATTGCCGGC |
| alphaMHC | CTCACTTGAAGGACACCCAGCTCCAGC |

Primers

Oct-4 Promoter
F-5′-GTGAGGTGTCGGTGACCCAAGGCAG-3′
R-5′-GGCGAGCGCTATCTGCCTGTGTC-3′
Bisulfite converted Oct4 promoter
1F-5′-GAAGGGGAAGTAGGGATTAATTTT-3′
1R-5′CAACAACCATAAACACAATAACCAA-3′
2F-5′-TAGTTGGGATGTGTAGAGTTTGAGA-3′
2R-5′-TAAACCAAAACAATCCTTCTACTCC-3′
3F-5′-AAGTTTTTGTGGGGGATTTGTAT-3′
3R-5′-CCACCCACTAACCTTAACCTCTA-3′

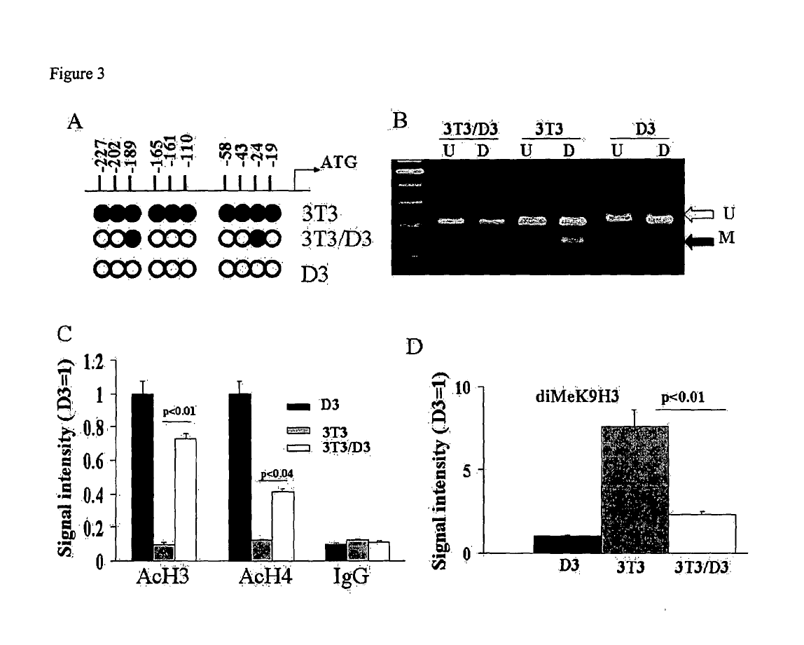

PCR products were directly sequenced. As depicted in FIG. 3A, upon bisulphite treatment, all 10 CpG sites in D3 cells were converted from C to T, indicating the unmethylated status of Oct4 promoter in this murine ESC cell line (open circles). All 10 sites were methylated in 3T3 cells (closed circles). In contrast, treatment of 3T3 cells with D3 extracts induced DNA de-methylation at 8/10 CpG residues. D3-extract induced Oct4 promoter demethylation in the 3T3/D3 cells was independently corroborated by restriction enzyme analysis. In the Oct4 promoter region, there is one HpyCH4IV (methylated CpG specific restriction enzyme) site at −202. The DNA methylation status of the −202 site was analyzed by HpyCH4IV restriction analysis in D3, 3T3 and 3T3/D3 cells. A ˜250 bp promoter region including site −202 of mouse Oct4 was amplified by PCR from the bisulphite treated genomic DNA from all three cell types. As shown in FIG. 3B, the PCR product was not digested with HpyCH4IV in D3 cells, indicating that the genomic DNA of D3 cells was unmethylated at this particular Oct4 promoter site. In contrast, the PCR product was readily digested in 3T3 cells indicating methylation of Oct4 −202 site. Interestingly, the PCR product from 3T3/D3 cells was resistant to digestion by HypCH4IV, suggesting that treatment of 3T3 cells with D3 extracts induced de-methylation of CpG sites thereby reversing the repression of Oct4 mRNA expression observed in 3T3 cells. DNA methylation/demethylation dependent gene suppression/activation is coupled with modifications to the histone proteins, which together lead to chromatin remodeling and new patterns of gene expression. To determine whether the D3-extract induced epigenetic changes in 3T3 cells, the acetylation of histones (H) 3 and 4 and methylation of histone 3 protein and its interaction with Oct4 promoter was analysed using Chromatin Immunoprecipitation (ChIP) assays (R. Kishore et al., J Clin Invest. 115, 1785 (2005). ChIP was performed using anti-acetylated H3 and H4 antibodies or anti-IgG antibodies. The Oct4 promoter was amplified from immunoprecipitated chromatin DNA by PCR. ChIP analyses showed that the promoter of Oct4 had increased acetylation of H3 and H4 (FIG. 3C) and decreased demethylation of lysine 9 of histone 3 (FIG. 3D) in 3T3/D3 cells compared to 3T3 cells. Without wishing to be bound by theory, these data indicate that D3-extract induced de-differentiation and nuclear re-programming of 3T3/D3 cells was mediated, at least in part, by chromatin remodeling leading to the activation of Oct4. The ChIP data indicates an association of acetylated H3 and H4 with Oct4 promoter following ESC-extract treatment of 3T3 cells. This finding is consistent with data showing that DNA methylation causes chromatin condensation through the formation of a protein complex consisting of methyl-binding protein, histone deacetylase, and repressor protein at the methylated CpG sites (P. L. Jones et al., Nat Genet. 2, 187 (1998) 1 X. Nan et al., Nature. 393, 386 (1998); H. H. Ng, P. Jeppesen, A. Bird, Mol Cell Biol. 4, 1394 (2000).

Considering that DNA methylation is involved in various biological phenomena, such as tissue-specific gene expression, cell differentiation, X-chromosome inactivation, genomic imprinting, changes in chromatin structure, and tumorogenesis, it is likely that changes in Oct4 promoter CpG methylation by exposure of 3T3 cells to ESC extracts is one of the epigenetic events underlying de-differentiation of 3T3 cells. Evidence present in the literature also supports this conclusion. In mice, Oct4, is expressed in the oocyte and preimplantation embryo but is later restricted to the inner cell mass of the blastocyst and in the derived ESC. Thus, Oct4 expression in mouse embryos is restricted to totipotent and pluripotent cells. In Oct4-deficient embryos, the inner cell mass loses pluripotency and the trophoblast cells no longer proliferate to form the placenta. Furthermore, reduction in Oct4 gene expression led to the trans-differentiation of ES cells into other cell types, demonstrating that suppression of the Oct4 gene is important for the determination of the potency of these stem cells. Moreover, Oct4 is one of the many embryonic genes known to be regulated by DNA methylation. Chromatin structure modification by histone acetylation is also involved in the epigenetic regulation of the Oct4 gene.

To gain further insight into the changes in gene expression patterns in reprogrammed 3T3/D3 cells, global gene-expression profiles of D3, 3T3 and 3T3/D3 and one single cell derived clone from 3T3/D3 cells was carried out using Affymetrix mouse genome 2A gene chips. Differentially expressed genes between the three cell types 3T3, D3, and 3T3/D3 were determined by a simple one-way ANOVA performed on the RMA expression values of each probe set, using the R package limma (G. K. Smythm et al., (eds.), Limma: linear models for microarray data. Bioinformatics and Computational Biology Solutions using R and Bioconductor, Springer, New York, pages 397-420 (2005)). A multiple testing adjustment (Y. Benjamini and Y. Hochberg, Journal of the Royal Statistical Society Series B, 57, 289 (1995)) was performed on the resulting statistics to adjust the false discovery rate. Differentially expressed probes with adjusted p-value <0.001 and a fold-change of greater than 2 (absolute log fold change of >than 1) were extracted for further inspection. This resulted in 3,286 probes with statistically significant differential expression between cell types 3T3 and 3T3/D3 including the significant up-regulation of ESC specific genes Oct4, nanog, Dppa 3 and 5, Slc2a3, zfp296, and kit, histones 1 and 2 and histone deacelylase etc.

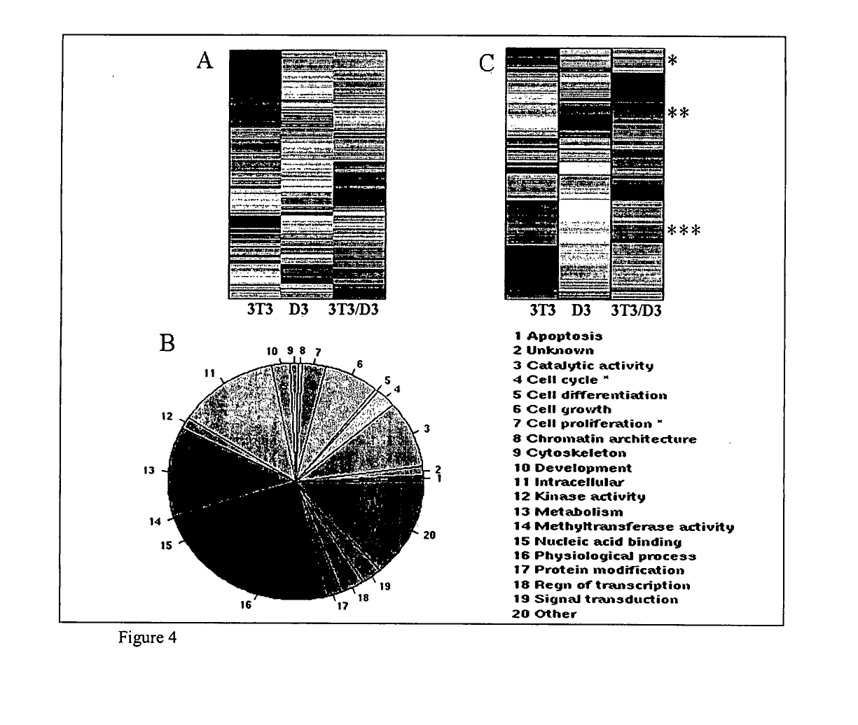

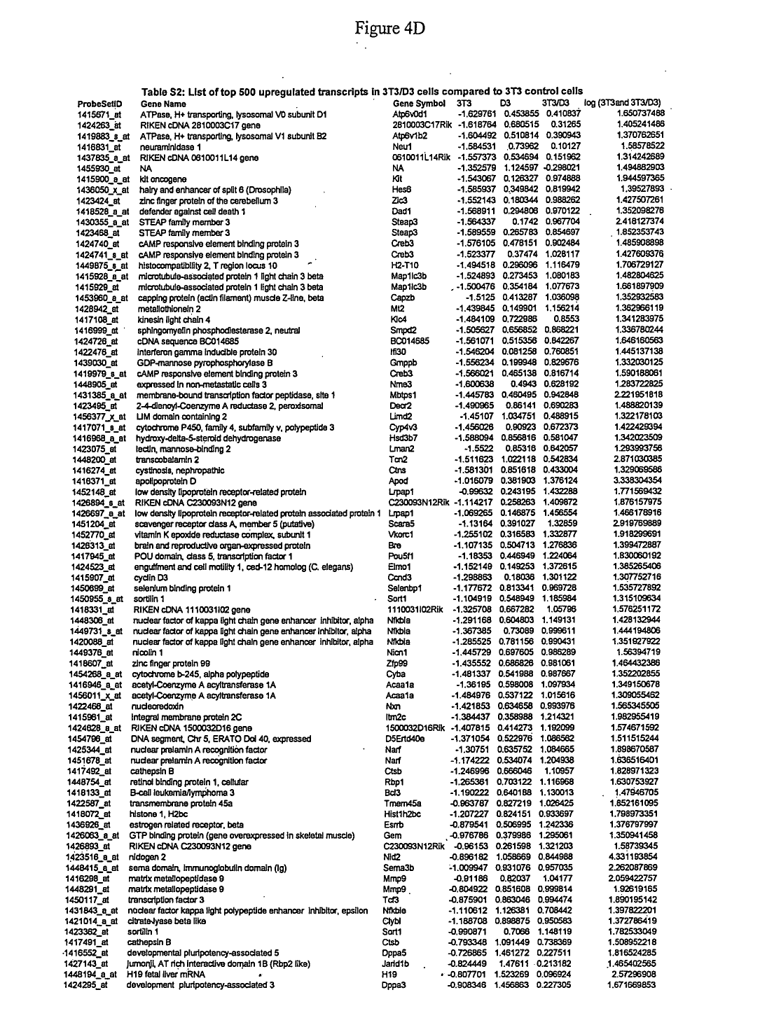

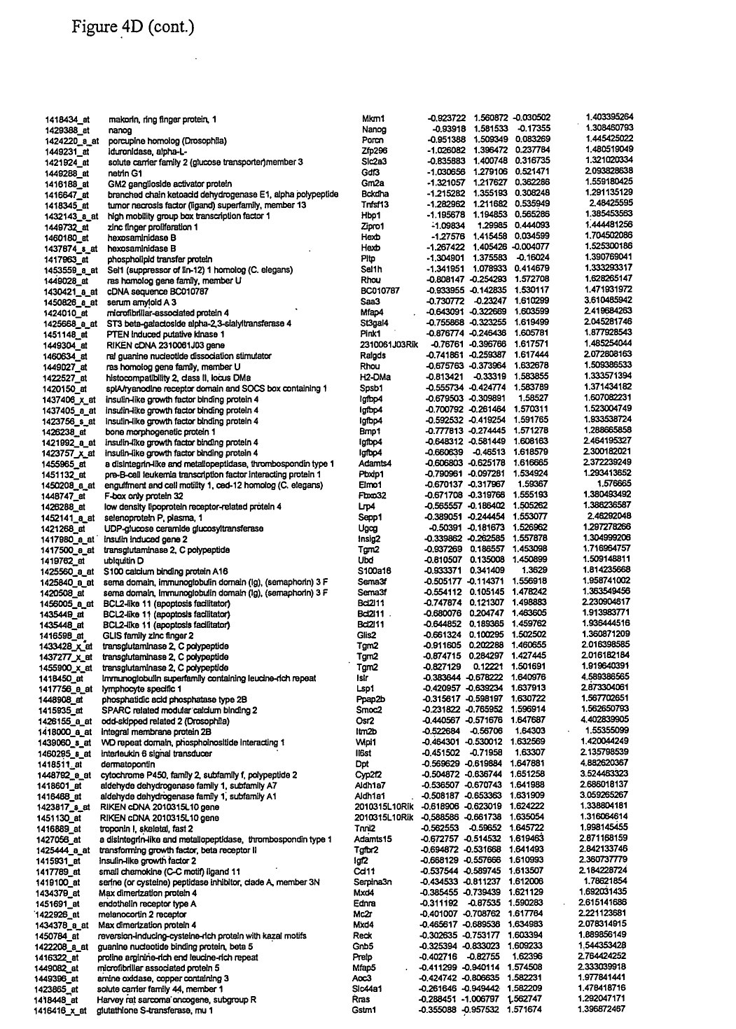

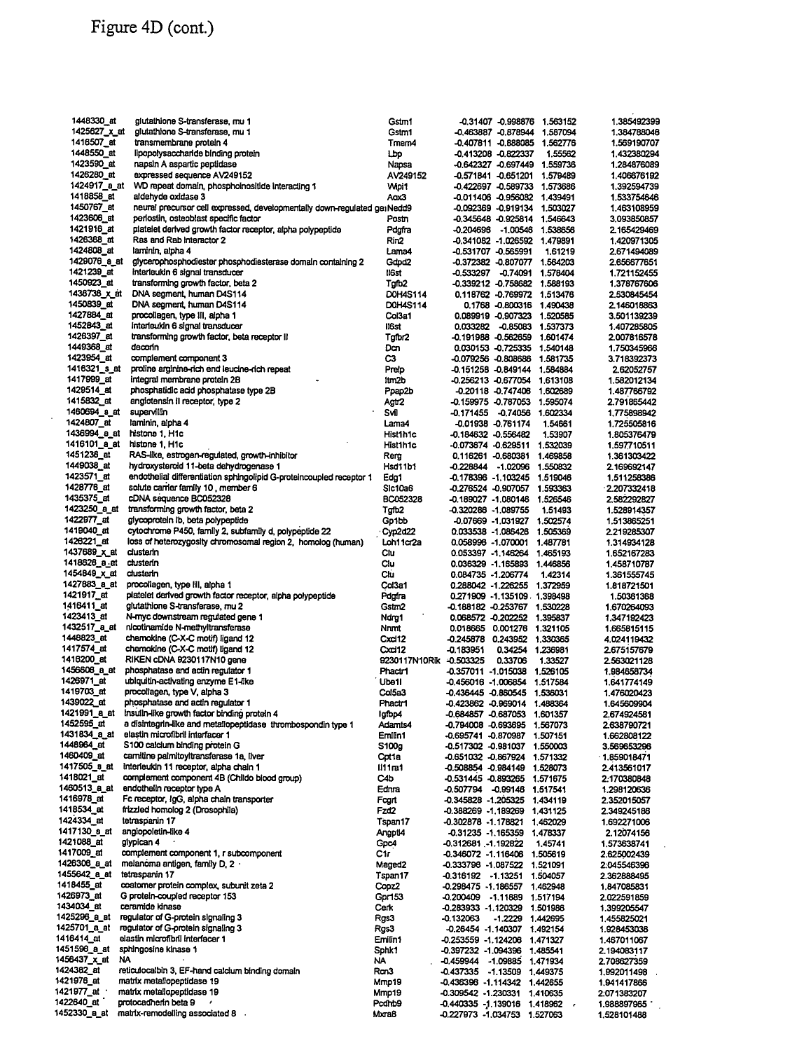

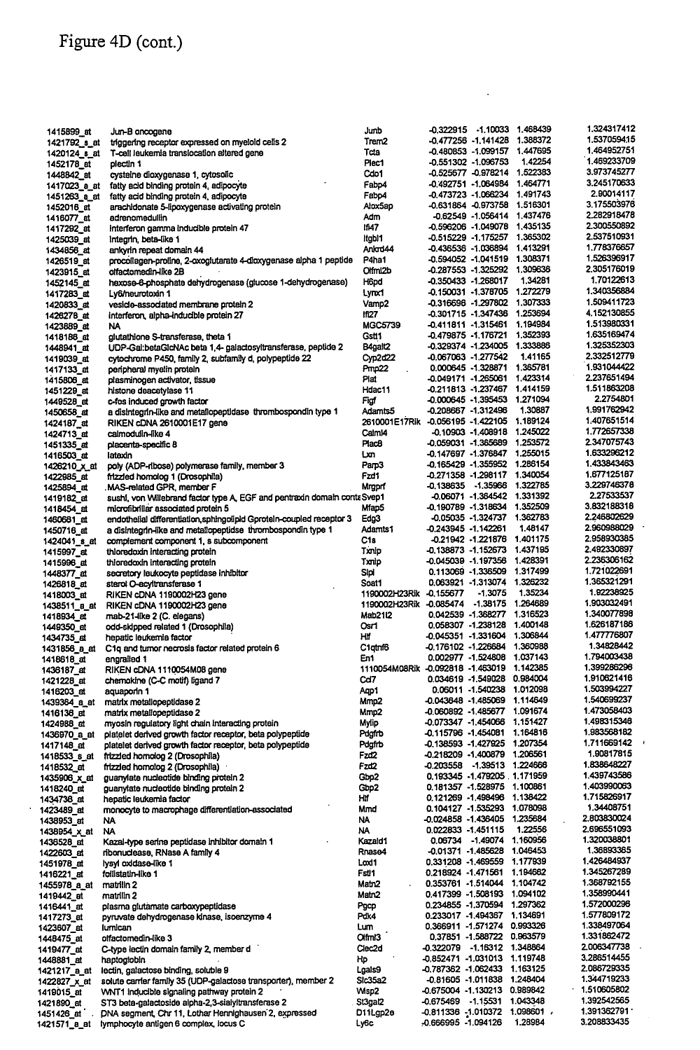

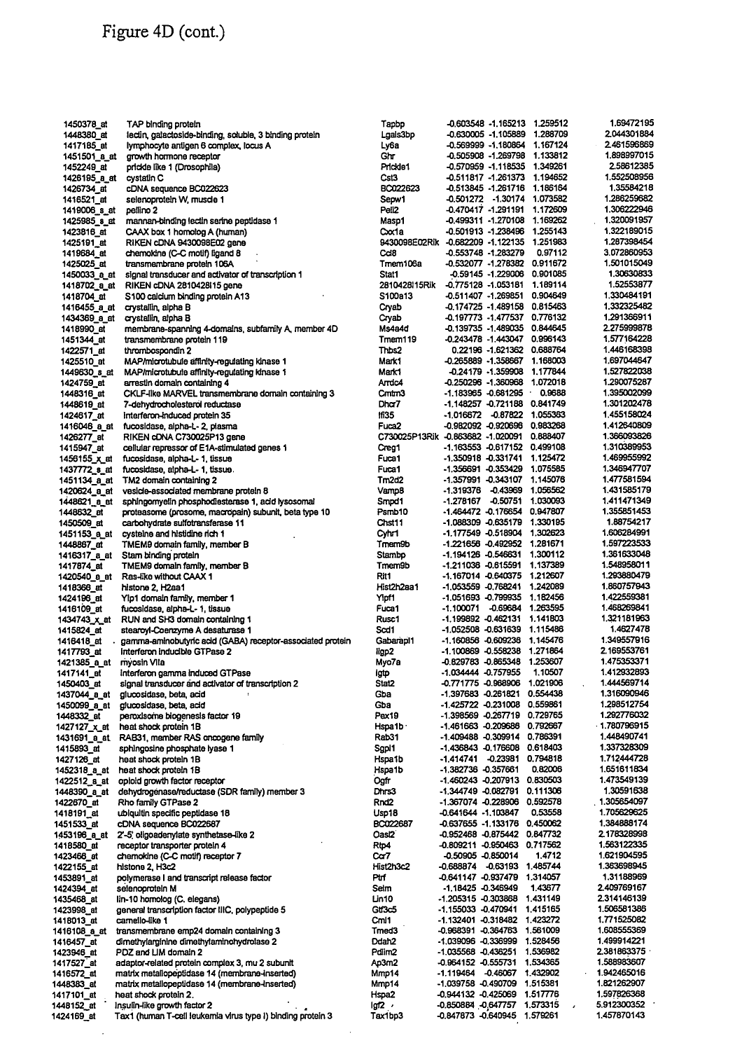

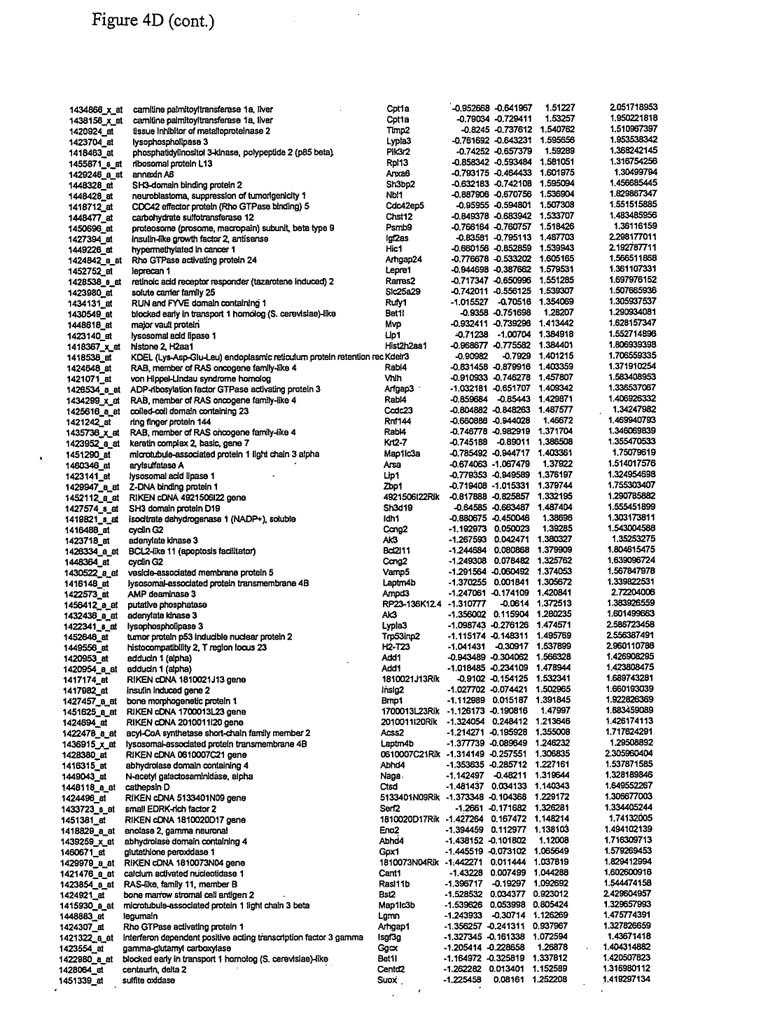

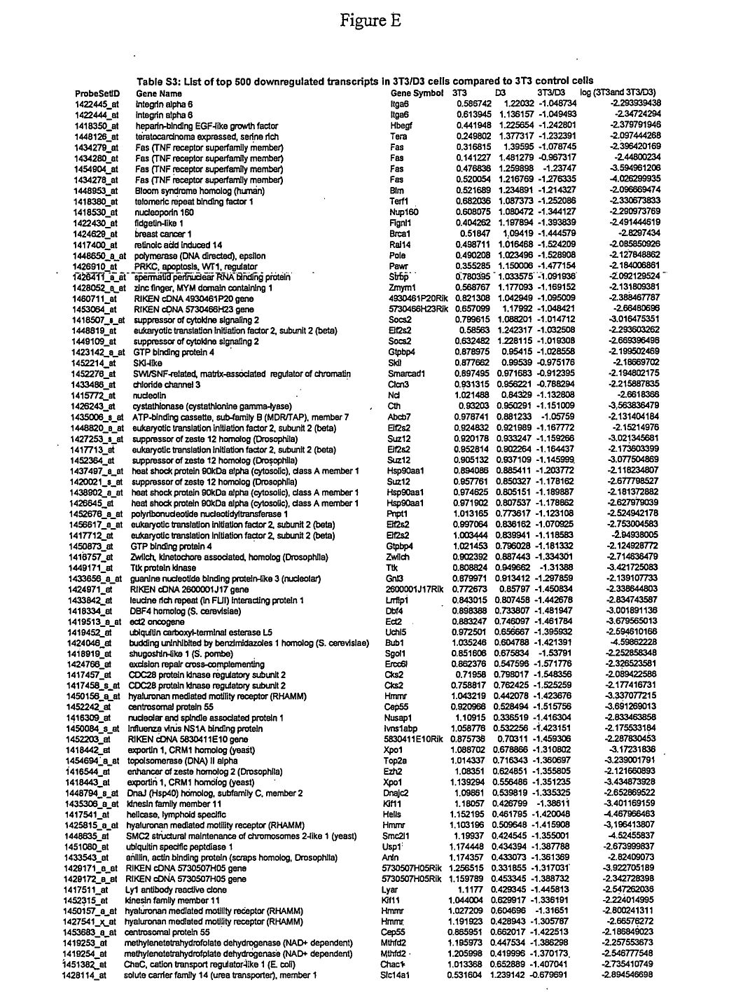

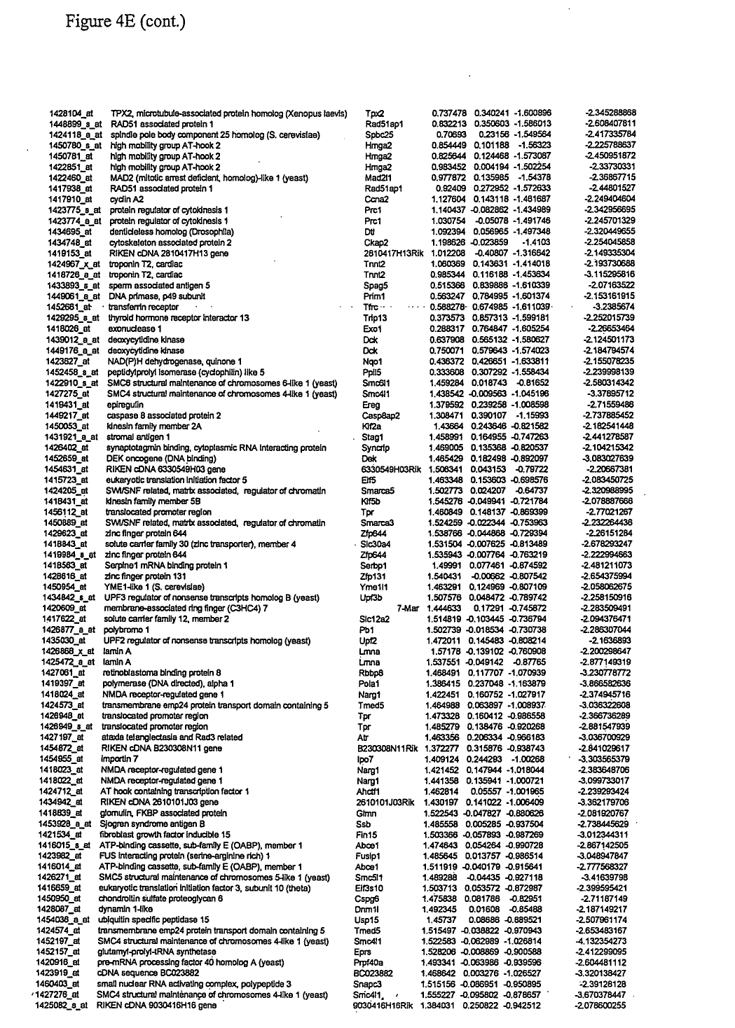

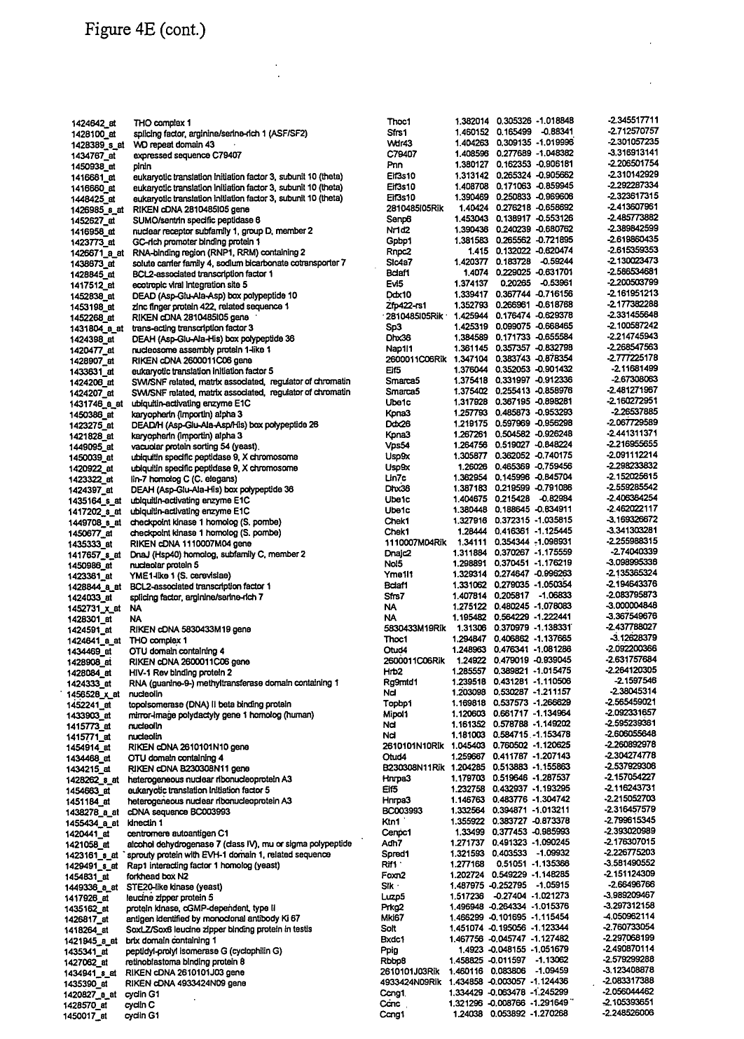

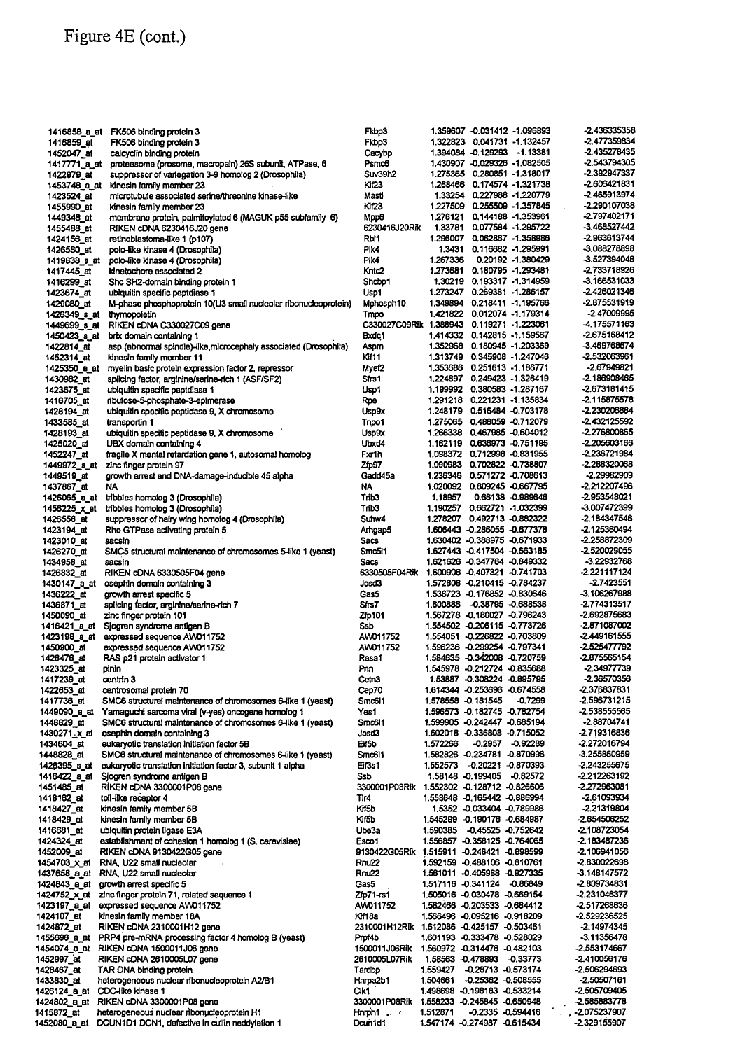

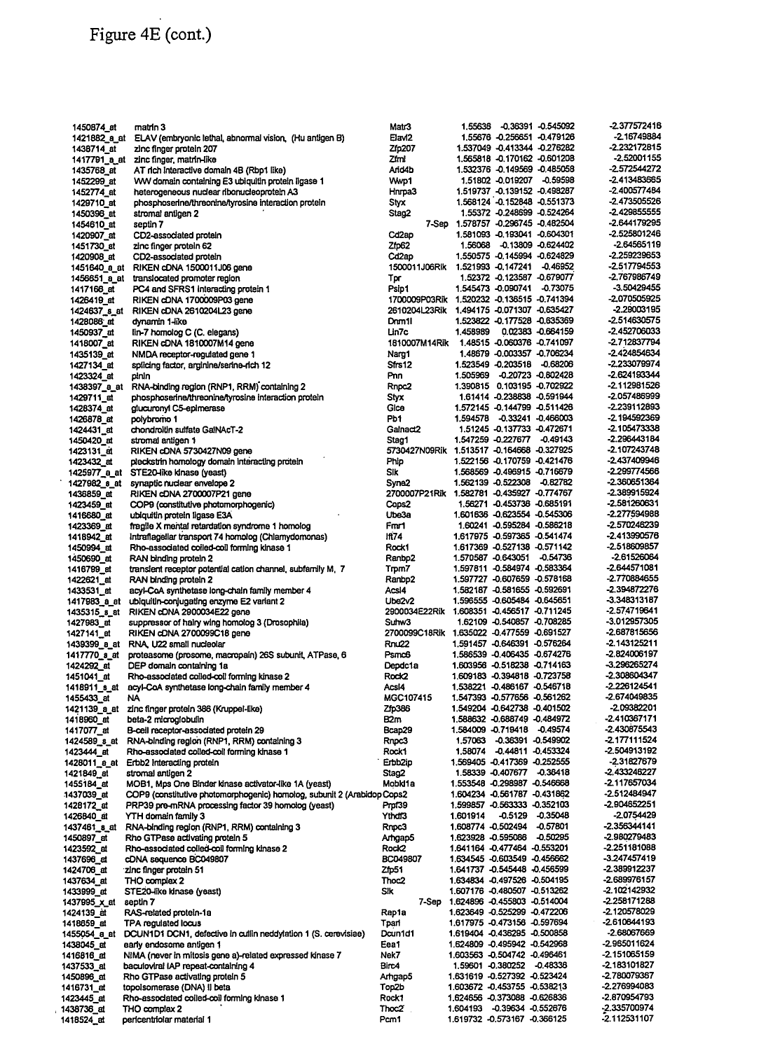

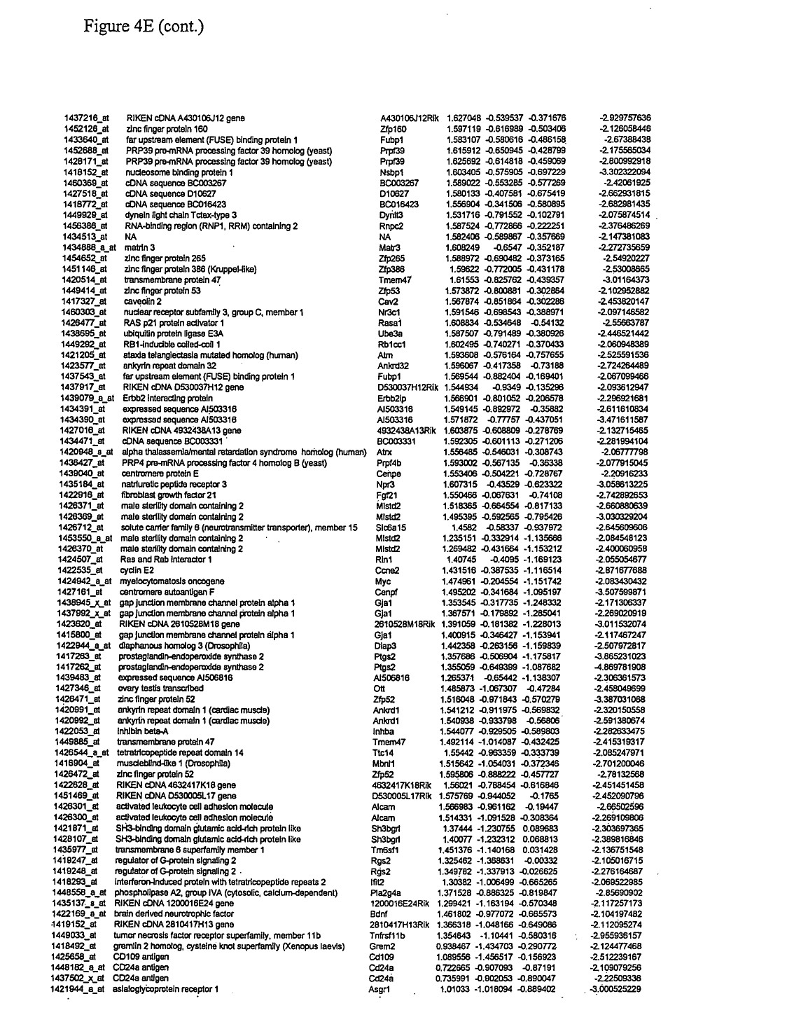

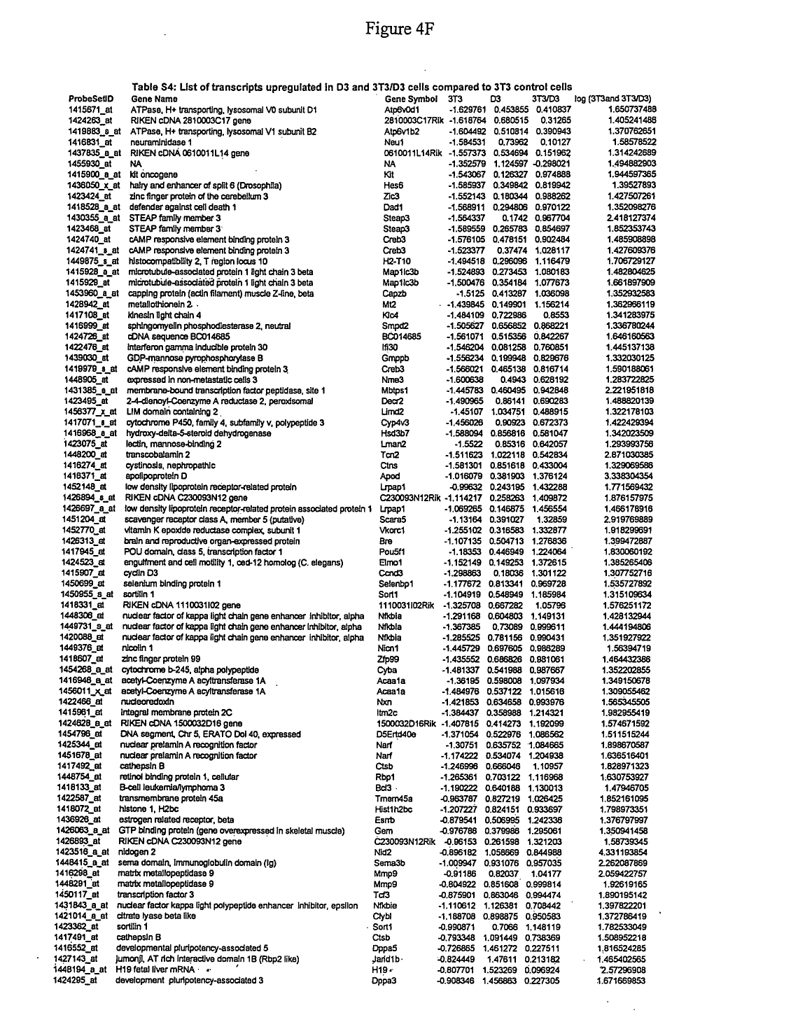

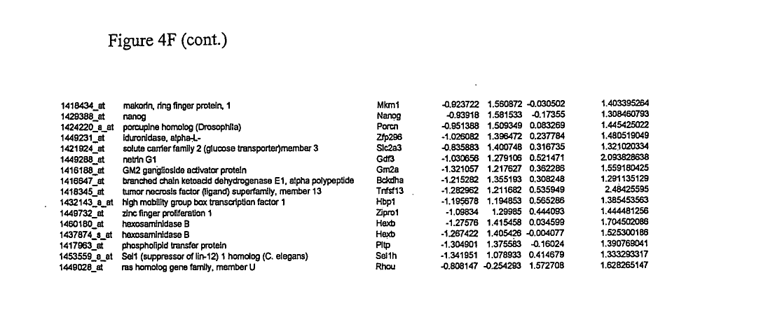

Genes significantly down-regulated in 3T3/D3 cells compared to 3T3 cells included lmna, cyclins A2, C and G1, tlr4, fas, hsp90prc1, SOCS2 etc. Hierarchical clustering, using the Pearson correlation coefficient and average agglomeration method was performed on the 3,286 genes (2187 down-regulated genes and 1099 up-regulated genes) that were differentially expressed between 3T3 and D3/3T3 cells. The heatmap in FIG. 4A of z-scored probes illustrates this clustering, in which z-scores (subtraction of mean and division by standard deviation of normalized values) were computed for each probe across all twelve arrays. The 3,286 genes found to be differentially expressed between 3T3 and 3T3/D3 cell types were categorized with respect to functional group (FIG. 4B) as per the software EASE (http://david.niaid.nih.gov/david/ease.htm). Two categories, cell cycle and cell proliferation, were found to be statistically over-represented (Bonferroni corrected p-value p<0.05) using the Fisher exact test via the software package EASE. Heatmap of the top 500 up-regulated and top 500 down-regulated z-scored probe sets in 3T3/D3 cells compared to 3T3 cells, is shown in FIG. 4C and the annotations of such genes are listed in FIGS. 4D and 4E, respectively. A list of genes showing significant up-regulation exclusively in D3 and 3T3/D3 cells as compared to 3T3 cells is depicted in FIG. 4F.

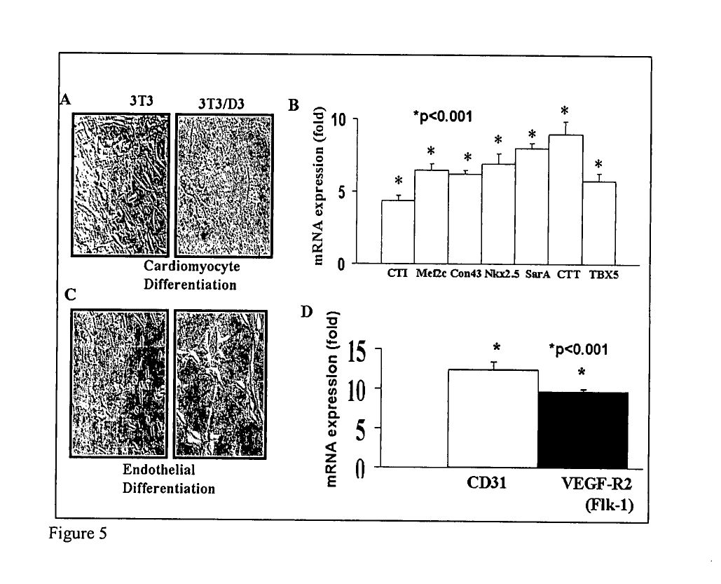

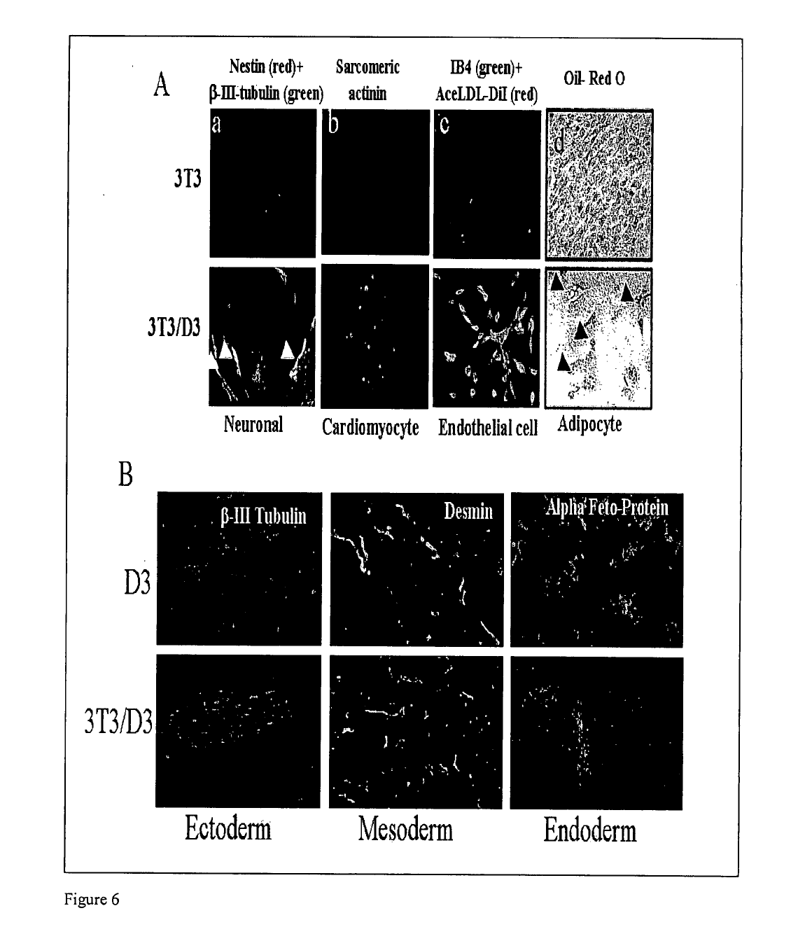

The differentiation potential of de-differentiated 3T3/D3 cells to multiple cell types was assessed in vitro and in vivo. The potential of 3T3/D3 cells to form cardiomyocytes was assayed in vitro. 3T3/D3 cells were cultured under conditions conducive for cardiomyocyte (CMC) and endothelial cell (EC) differentiation. As shown in FIGS. 5A and 5C) 3T3/D3 cells elicited changes in cell morphology consistent with CMC and EC phenotype, which was corroborated by marked increase in mRNA expression of CMC (FIG. 5B) and EC (FIG. 5D) specific genes. Further evidence in support of the CMC and EC differentiation of 3T3/D3 cells is provided by the finding that the cells expressed EC and CMC specific markers in immunofluorescence assays for sarcomeric actinin (FIG. 6A, panel b), which is a CMC-specific marker and for the EC specific markers Isolectin B4 (IB4). In addition, 3T3/D3 cells showed uptake of DiI labeled acetylated-LDL (FIG. 6A, panel c), which is also consistent with an EC cell fate. To determine whether 3T3/D3 cells were capable of differentiating towards multiple cell types, neuronal cell and adipocyte specific protein expression was examined. 3T3/D3 cells were cultured under conditions suitable for neuronal or adipocyte fate induction. Results are shown in (FIG. 6A, panels a, d)

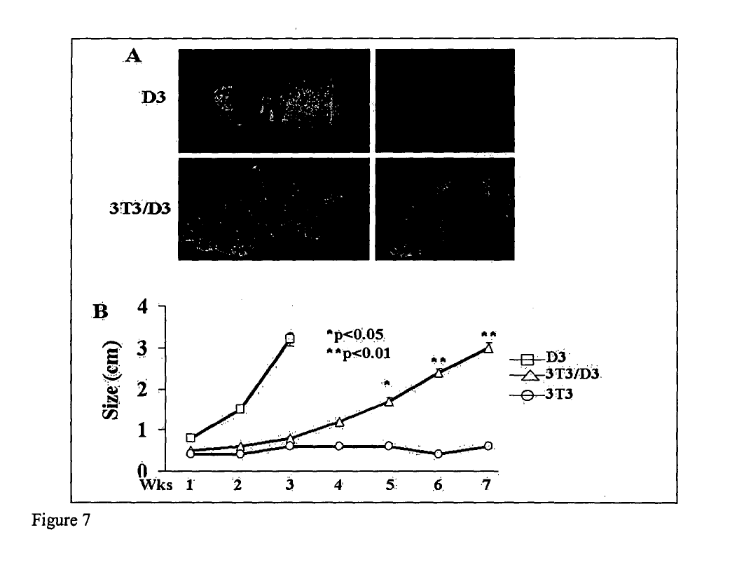

In addition, the multilineage differentiation of 3T3/D3 cells was examined by assaying for teratoma formation. 3T3/D3 cells were injected subcutaneously into SCID mice. The injected cells reproducibly formed teratomas. The formation of teratomas occurred with slower kinetics relative to teratoma formation in SCID mice injected with D3 cells (FIGS. 7A, 7B). As shown in FIG. 6B, 3T3/D3 cells differentiated into representative cell types of all 3 germ layers, confirmed by immunofluorescence staining for the expression of βIII-tubulin (ectodermal), desmin (mesodermal) and α-fetoprotein (endodermal).

The trans-differentiation of 3T3/D3 cells into CMC and EC observed in vitro, prompted examination of whether ischemic myocardium could be repaired by transplantation of these cells in a model of acute myocardial infarction (AMI). Mice underwent surgery to induce AMI by ligation of the left anterior descending coronary artery, as described (A. Iwakura et al., Circulation. 113, 1605 (2006)). Animals were sub-divided into 3 groups (10 mice in each group), and received intramyocardial injection of 5×10⁴ lentiviral-GFP transduced (to track transplanted cells up to 4 weeks, in vivo) 3T3/D3 or 3T3 control cells or saline, respectively, in a total volume of 10 μl at 5 sites (basal anterior, mid anterior, mid lateral, apical anterior and apical lateral) in the peri-infarct area. Left ventricular function was assessed by transthoracic echocardiography (SONOS 5500, Hewlett Packard) as described (A. Iwakura et al., Circulation. 113, 1605 (2006); Y. S. Yoon et al., J Clin Invest. 111, 717 (2003)). A physiological assessment of left ventricular (LV) function after AMI was performed in all groups of mice at basal level before surgery and on days 7, 14 and 28, post-AMI. Left ventricular end-diastolic areas (LVEDA) were similar in the 3T3 cell and saline groups before and at all time points after AMI (FIG. 8A, black (3T3/D3) and grey lines (Saline and 3T3/3T3), respectively). In contrast, mice treated with the 3T3/D3 cells had less ventricular dilation when assayed by echocardiography (FIG. 8A black line, p<0.05 in 3T3/D3 vs. control groups) when treated with 3T3/D3 cells beginning at 1 week post AMI then mice treated with 3T3 cells. Fractional shortening (FS), an indicator of contractile function, was evaluated on day 7, 14 and 28 in all groups. As shown in FIG. 8B FS was consistently depressed following AMI in control mice receiving saline and control 3T3 cells relative to pre-surgery (basal) FS. Mice treated with 3T3/D3 cells following AMI showed significantly improved FS at all time points tested (at 4 weeks post-surgery, p<0.01 vs. saline group and p<0.05 vs. control 3T3 cell treated group). The individual values for evaluated LV function parameters at various time points are shown in Table 2 (below).

TABLE 2
Hemodynamic parameters in mice receiving cell therapy

| | | | | After MI | | |
|---|---|---|---|---|---|---|
| parameters | n | group | Before MI | 1 week | 2 weeks | 4 weeks |
| LVEDA, mm2 | 8 | 3T3/D3 | 11.7 ± 0.7 | 13.6 ± 0.6** | 15.0 ± 0.6 | 14.6 ± 6.6* |
| | 6 | 3T3/3T3 | 12.2 ± 0.4 | 16.4 ± 0.4 | 16.6 ± 0.8 | 18.0 ± 1.1 |
| | 5 | Saline | 13.3 ± 0.6 | 14.3 ± 1.7 | 13.5 ± 1.6 | 17.6 ± 2.9 |
| LVESA, mm2 | 8 | 3T3/D3 | 5.1 ± 0.3 | 7.2 ± 0.4*** | 7.8 ± 0.5* | 7.8 ± 0.6*** |
| | 6 | 3T3/3T3 | 5.0 ± 0.2 | 10.5 ± 0.6 | 10.3 ± 0.8 | 11.9 ± 0.8* |
| | 5 | Saline | 6.8 ± 0.5 | 9.6 ± 1.4 | 9.5 ± 1.7 | 12.9 ± 1.9 |
| LV area FS, % | 8 | 3T3/D3 | 55.8 ± 1.3 | 46.9 ± 2.9* | 48.3 ± 1.6* | 46.6 ± 3.2* |
| | 6 | 3T3/3T3 | 55.0 ± 2.5 | 36.1 ± 2.8 | 38.2 ± 3.7 | 34.1 ± 2.4 |
| | 5 | Saline | 53.7 ± 4.9 | 32.9 ± 2.1 | 30.8 ± 1.7 | 30.8 ± 0.9 |
| LVDd, mm | 8 | 3T3/D3 | 282 ± 3.3 | 320 ± 6.1** | 337 ± 7.6*** | 355 ± 7.6* |
| | 6 | 3T3/3T3 | 291 ± 3.2 | 355 ± 7.4 | 383 ± 6.4 | 391 ± 11.0* |
| | 5 | Saline | 284 ± 3.0 | 314 ± 4.0 | 318 ± 3.4 | 397 ± 15.5 |
| LVDs, mm | 8 | 3T3/D3 | 100 ± 5.4 | 130 ± 6.1*** | 140 ± 10.6** | 142 ± 6.2** |
| | 6 | 3T3/3T3 | 100 ± 3.4 | 166 ± 9.5 | 177 ± 7.7 | 187 ± 9.2 |
| | 5 | Saline | 109 ± 6.1 | 113 ± 14.0 | 183 ± 25.2 | 233 ± 16.0 |
| LVFS, % | 8 | 3T3/D3 | 64.6 ± 1.7 | 59.5 ± 1.8* | 58.7 ± 2.3 | 58.2 ± 2.2* |
| | 6 | 3T3/3T3 | 65.5 ± 1.2 | 53.2 ± 1.9 | 53.7 ± 1.6 | 52.2 ± 1.3 |
| | 5 | Saline | 61.6 ± 2.2 | 64.0 ± 4.6 | 42.6 ± 7.6 | 41.3 ± 1.7 |

LVEDA: Left ventricular end-diastolic Area,
LVESA: Left ventricular end-siastolic Area,
FS: Fractional shortening,
LVDd: Left ventriculat Diastolic dimension,
LVDs: Left ventricular systolic dimension.
*p < 0.05;
**p < 0.01;
***p < 0.001

Improvement in post-AMI physiological heart function in mice that received D3-extract treated 3T3 cell transplanted was also observed when treated hearts were histologically evaluated. Immunofluorescence staining of myocardial sections was performed to determine CMC and EC differentiation of the transplanted (GFP⁺) cells and to determine myocardial proliferation (ki67 staining) and apoptosis (TUNEL) (Y. S. Yoon et al., J Clin Invest. 111, 717 (2003). Fibrosis was assayed by embedding heart tissues sections in paraffin, staining the tissue for elastic tissue/trichrome (ET), and measuring the average ratio of the external circumference of fibrosis area to LV area. As shown in FIG. 8C, the fibrosis area in mice hearts receiving either saline or control 3T3 cells was significantly larger than that observed in mice that received 3T3/D3 cells (p<0.001). Tissue sections were also stained with BS1 lectin to determine the capillary density at the border zone of the infracted myocardium. Significantly higher capillary density was observed in mice receiving 3T3/D3 cells than in mice receiving control 3T3 cells or saline (FIG. 8D, p<0.01).

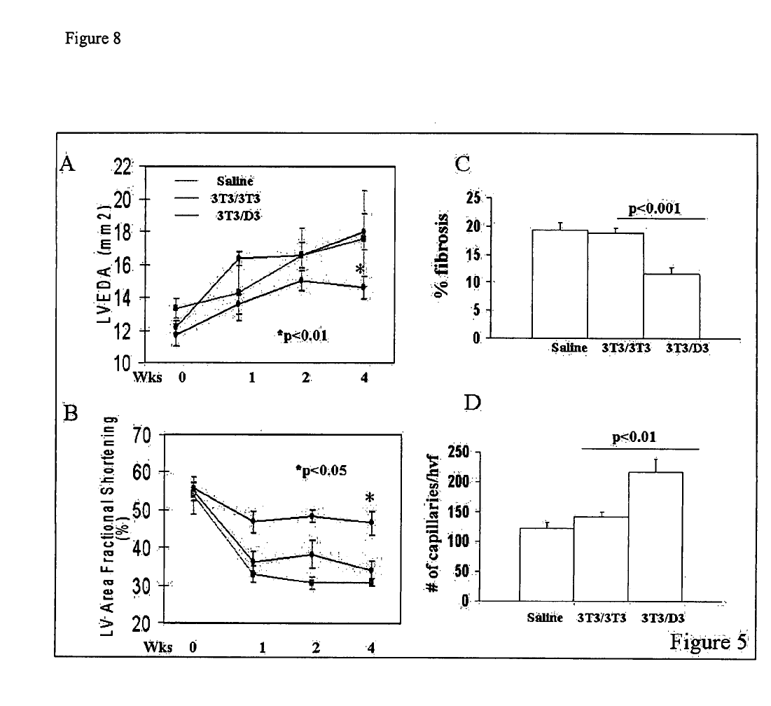

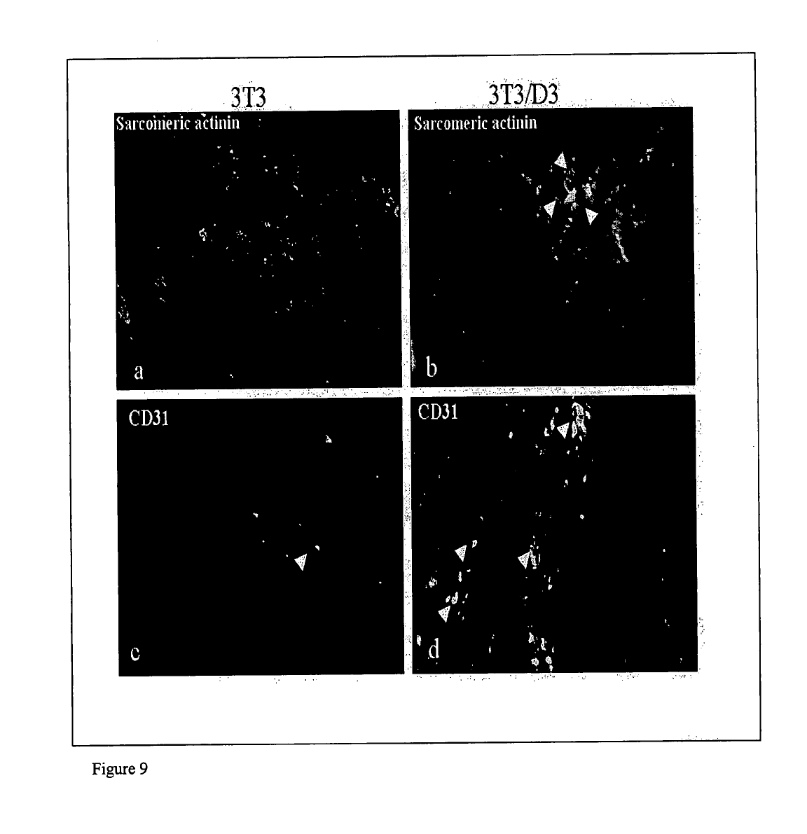

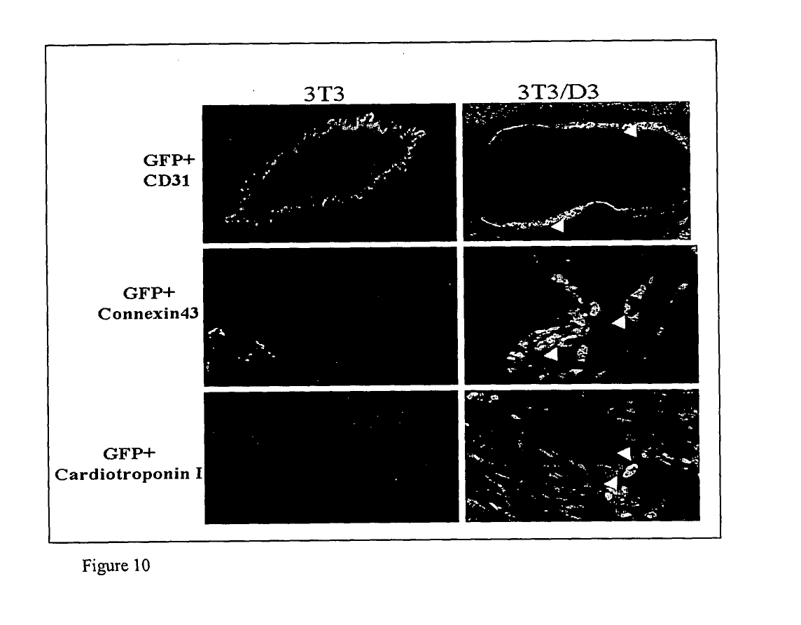

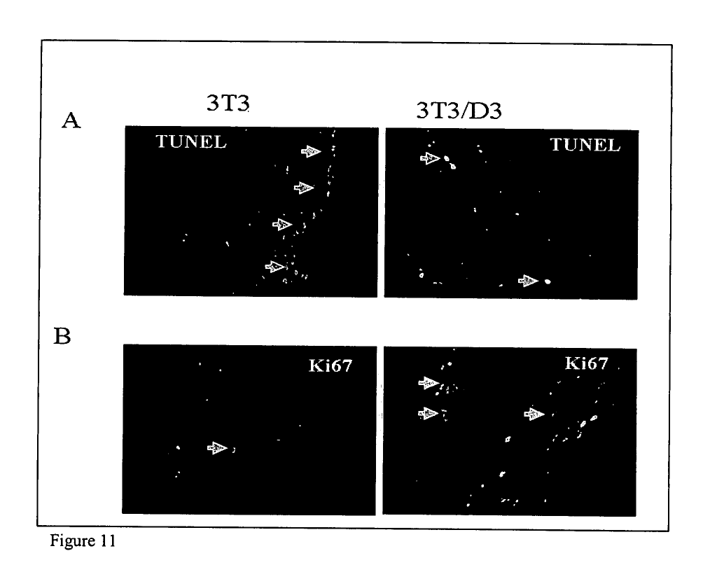

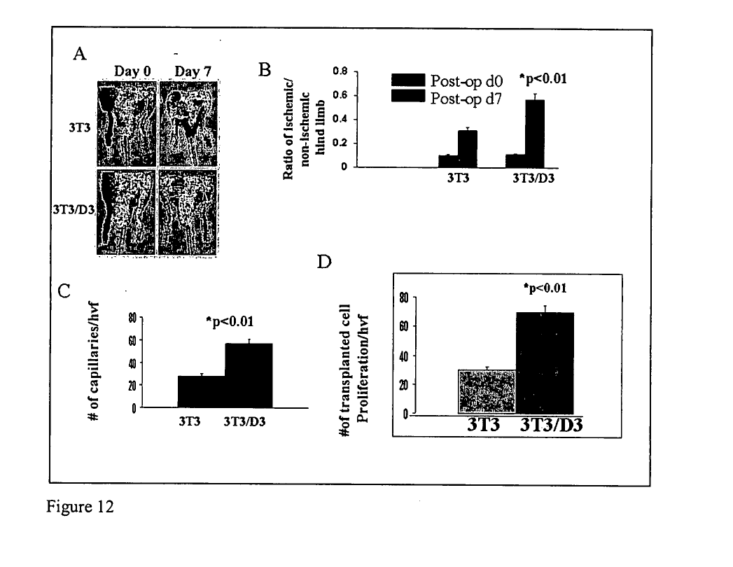

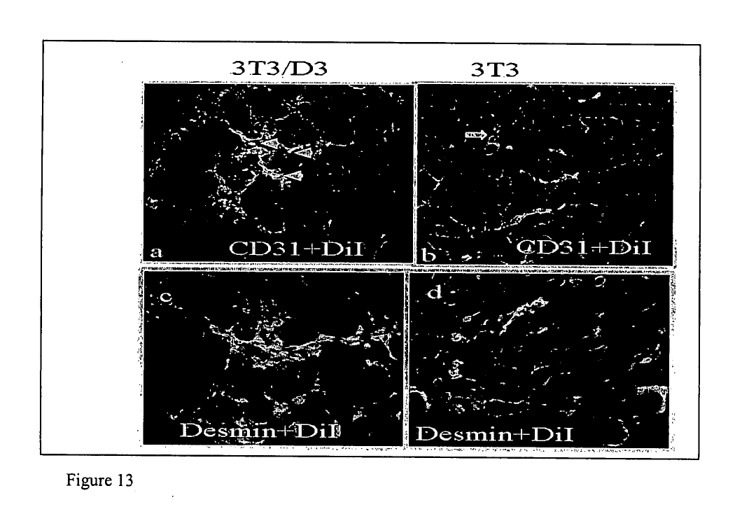

To evaluate the CMC differentiation of the transplanted cells in the myocardium, tissue sections were stained with a specific CMC marker, sarcomeric actinin, and the GFP⁺ cells (3T3 or 3T3/D3; green) co-expressing sarcomeric actinin (red) were visualized as double positive (yellow) cells in the merged images. As shown in FIG. 9A-9D, GFP⁺ sarcomeric actinin double positive cells (light fluorescence) were observed in mice treated with 3T3/D3 cells suggesting that some of the transplanted cells differentiated into CMC lineage in vivo, while no evidence of CMC differentiation was observed for transplanted 3T3 cells. Immunofluoresence staining of additional CMC specific markers, connexin43 and cardiotroponin I, further corroborated CMC differentiation of 3T3/D3 cells (FIG. 10). Similar observations were revealed when EC differentiation of transplanted cells was investigated. As shown in FIG. 10, GFP+CD31 double positive cells were observed in myocardial sections obtained from mice transplanted with 3T3/D3 cells, while sections obtained from control 3T3 transplanted hearts did not show the evidence for EC differentiation. Some of the transplanted 3T3/D3 cells physically incorporated into the vasculature (FIG. 10, top panel, arrow heads). The apoptotic and proliferating myocardial cells were also quantified following cell transplantation. The number of apoptotic cells, as evident from TUNEL+ cells, were significantly higher in myocardial sections of mice treated with control 3T3 as compared to those treated with 3T3/D3 cells (FIG. 11A; 18+ 2.3 TUNEL+ cells/ high visual field in control 3T3 group vs. 5+ 1.2 TUNEL+ cells/high visual field in 3T3/D3 cell group; p<0.01). A higher number of proliferating cells, (nuclei stained positive for Ki67) were also observed in the myocardial sections from 3T3/D3 treated mice as compared to control 3T3 treated mice (FIG. 11B, p<0.05). The therapeutic effect of 3T3/D3 cells on physiological blood flow recovery and neo-vascularization in a mouse model of surgically induced hind limb ischemia was also examined. Transplantation of 3T3/D3 cells in the ischemic hind limbs of mice resulted in robust physiological blood flow recovery on day 7 post-injury, compared to mice that were transplanted with 3T3 cells (FIGS. 12A, 12B), as measured by laser Doppler perfusion imaging. Additionally, 3T3/D3 transplanted ischemic limbs displayed a significantly higher number of capillaries, suggesting enhanced neo-vascularization as well a higher proliferation of transplanted 3T3/D3 cells, in vivo (FIGS. 12C, 12D). Furthermore, transplanted 3T3/D3 cells expressed EC (CD31) and muscle cell (desmin) markers indicating in vivo differentiation into cells of these two lineages (FIG. 13).

The goal of therapeutic cloning is to produce pluripotent stem cells with the nuclear genome of the subject and induce the cells to differentiate into replacement cells, for example, cardiomyocytes, for repairing damaged heart tissue. Reports on the generation of pluripotent stem cells (J. B. Cibelli et al., Nat Biotechnol. 16, 642 (1998); M. J. Munsie et al., Curr Biol. 10, 989 (2000); T. Wakayama et al., Science. 292, 740 (2001)) or histocompatible tissues (R. P. Lanza et al., Nat Biotechnol. 20, 689 (2002)) by nuclear transplantation, and on the correction of a genetic defect in cloned ESCs (W. M. Rideout et al., Cell. 109, 17 (2002)) suggest that therapeutic cloning could in theory provide a source of cells for regenerative therapy. Recent evidence on the efficacy of human therapeutic cloning, however, underscores the difficulties associated with the generation of human ESC lines for therapeutic purposes. Moreover, a number of limitations may hinder the strategy of therapeutic cloning for future clinical applications. Extremely low efficiency of somatic nuclear transfer is a major concern. Analysis of the literature on mouse SNT derived ESC lines raises concerns about the feasibility and relevance of therapeutic cloning, in its current embodiment, for human clinical practice. Nuclear transfer is unlikely to be much more efficient in human than in mouse. Optimistically, ≈100 human oocytes would be required to generate customized ESC lines for a single individual. Including the complexity of the volunteer reimbursement and oocyte retrieval procedure, the cost of a human oocyte could be ≈$1,000-2,000 in the U.S. Thus, to generate a set of customized ESC lines for an individual, the budget for the human oocyte material alone would be ≈$100,000-200,000. This prohibitively high sum might impede the widespread application of this technology in its present form. This limitation might be alleviated with oocytes from other species, but mitochondrial genome differences between species are likely to pose a problem. Another current challenge of therapeutic cloning is to overcome abnormalities encountered in cloned animals as these may reflect defects in cloned ESC. In spite of the production of cloned animals and ESC-like cells by nuclear transplantation, reports of unstable or abnormal gene expression patterns in cloned embryos and fetuses suggest incomplete reprogramming. Finally, ethical debate related to human oocyte manipulations add to the limitations. It is therefore desirable to develop alternative strategies to oocyte-dependent therapeutic cloning. The results described herein indicate that mESC extract-mediated reverse lineage commitment of terminally differentiated murine fibroblasts and re-differentiation of these reprogrammed cells support the feasibility of this approach and provide evidence that the stem-like cells obtained using this methodology are functionally competent for tissue repair.

This is the first study to demonstrate that mESC extracts can not only reprogram somatic cells towards multipotency but more importantly, de-differentiated somatic cells can trans-differentiate into endothelial, skeletal muscle and cardiomyocytes in vivo and repair damaged tissues, in experimental critical limb ischemia and acute myocardial infarction and critical limb ischemia models. The histological evidence is well supported by physiological data that shows significant improvements in left ventricular function and hind limb perfusion in AMI and HLI models, respectively. Taken together our biochemical, molecular and functional data provide an oocyte-independent approach for the generation of functional autologous multipotent cells from terminally differentiated somatic cells. Refinement of techniques and additional experimental data to elucidate applicability of this approach in primary somatic cells of different lineages may hold significant promise for future use of such generated cells in regenerative medicine, including cardiac repair and regeneration.

The results reported above, were obtained using the following materials and methods.

### Cell Culture

NIH3T3 Swiss-Albino fibroblasts (ATCC) were cultured in DMEM (Sigma-Aldrich) with 10% FCS, L-glutamine, and 0.1 mM β-mercaptoethanol. The D3-mESC were obtained from ATCC (CRL-1934™) and cultured in DMEM medium supplemented with 10% FBS, 1 mM Sodium pyruvate, 100 U/ml penicillin G, 100 μg/ml Streptomycin, 2 mM glutamine, 1 mM MEM nonessential amino acids, 50 μM 2-mercaptoethanol, 100 mM MTG and 10 ng/ml of LIF (Gibco BRL, Rockville, Md.), in A 5% CO₂ incubator at 37° C. D3 cells were cultured free of feeder cells and sub-cultured every 3-5 days with one medium change in between days.

### Cell Extracts

The mouse embryonic stem cells (mESC) and 3T3 cells extract was prepared as described by Taranger et al., Mol Biol Cell. 16: 5719 (2005), which is herein incorporated by reference. Briefly, the cells were washed in phosphate-buffered saline (PBS) and in cell lysis buffer (100 mM HEPES, pH 8.2, 50 mM NaCl, 5 mM MgCl₂, 1 mM dithiothreitol, and protease inhibitors), sediment at 10,000 rpm, re-suspended in 1 volume of cold cell lysis buffer, and incubated for 30-45 minutes on ice. Cells were sonicated on ice in 200-μl aliquots using a sonicator fitted with a 3-mm-diameter probe until all cells and nuclei were lysed, as judged by microscopy. The lysate was sediment at 12,000 rpm for 15 minutes at 4° C. to pellet the coarse material. The supernatant was aliquoted, frozen in liquid nitrogen and stored at −80° C. Protein concentration of the mESC and NIH3T3 cell extracts were determined by Bradford assay.

### Streptolysin-O (SLO)-Mediated Permeabilization and Cell Extract Treatment

The SLO-mediated permeabilization and extract treatment was performed as follows. NIH3T3 cells were washed in cold PBS and in cold Ca²⁺- and Mg²⁺-free Hank's balanced salt solution (HBSS) (Invitrogen, Carlsbad, Calif.). Cells were re-suspended in aliquots of 100,000-cells/100 μl of HBSS, or multiples thereof; placed in 1.5-ml tubes; and centrifuged at 1500 rpm for 5 minutes at 4° C. in a swing-out rotor. Sedimented cells were suspended in 97.7 μl of cold Hank's buffered salt solution (HBSS). Tubes were placed in a H₂O bath at 37° C. for 2 minutes, and 2.3 μl of SLO (Sigma-Aldrich) (100 μg/ml stock diluted 1:10 in cold HBSS) was added to a final SLO concentration of 230 ng/ml. Samples were incubated horizontally in a H₂O bath for 50 minutes at 37° C. with occasional agitation and set on ice. Samples were diluted with 200 μl of cold HBSS, and cells were sedimented at 1500 rpm for 5 minutes at 4° C. Permeabilization efficiency of >80% was obtained as assessed by monitoring uptake of a 70,000-Mᵣ Texas Red-conjugated dextran (50 μg/ml; Invitrogen). After permeabilization, NIH3T3 cells were suspended at 2,000 cells/μl in 100 μl of mESC extract or control NIH3T3 cells extract containing an ATP-regenerating system (1 mM ATP, 10 mM creatine phosphate, and 25 μg/ml creatine kinase; Sigma-Aldrich), 100 μM GTP (Sigma-Aldrich), and 1 mM each nucleotide triphosphate (NTP; Roche Diagnostics, Indianapolis, Ind.). The tube containing cells were incubated horizontally for 1 hr at 37° C. in a H₂O bath with occasional agitation. To reseal plasma membranes, the cell suspension was diluted with complete DMEM medium containing 2 mM CaCl₂ and antibiotics, and cells were seeded at 100,000 cells per well on a 48-well plate. After 2 hours, floating cells were removed, and plated cells were cultured in complete DMEM medium.

### Determination of De-Differentiation

De-differentiation of NIH3T3 following D3-cell extract treatment was determined by the induction of ESC markers both at the mRNA level by quantitative real time PCR and at the protein level by immuno-staining. Both self-extract treated and D3-extract treated 3T3 cells were cultured in the presence of 10 ng/ml of LIF for 10 days. The treated cells were then subcultured by plating 1×10⁶ cells per well for an additional 2 days in a 6-well culture plate. Total cellular RNA was harvested and the quantitative real-time RT-PCR was performed to determine mRNA expression of selected embryonic stem cell markers in self extract and D3-extract treated cells, as described previously (40). Primers used to amplify embryonic stem cell markers Nanog, SCF, SSEA1, Oct-4 and c-Kit are listed in table 1. Relative mRNA expression of target genes was normalized to the endogenous 18S control gene (Applied Biosystems). Enhanced expression of stem cell specific mRNAs was further corroborated by immunofluorescence protein staining of induced specific stem cell markers in NIH3T3 cells treated with D3 extract. For immuno-staining, both control and D3 extract treated NIH3T3 cells were cultured in medium in the absence and presence of LIF for 10 days. Then the cells were harvested and cultured 1×10⁴ cells per well in 4-well slides coated with 0.5% gelatin for another 2 days. The slides were stained with specific antibodies to stem cell markers, c-Kit and Oct-4. De-differentiation was also determined by the lamin B and lamin A/C (markers of soma) protein expression.

### In Vitro Cardiomyocytes (CMC) and Endothelial Cell (EC) Lineage Differentiation of D3-Extract Treated NIH3T3 Cells

To determine their transdifferentiating potency, dedifferentiated NIH3T3 cells and control cells were cultured in complete DMEM containing 5 ng/ml of LIF and 3 ng/ml of Bone morphogenic protein-2 (BMP2) in 6-well culture plates (1×10⁶ cells per well) and 4-well chamber slides (1×10⁴ cells per well) coated with 0.5% gelatin for 7 days. Total cellular RNA was harvested from 6-well culture plate and used to analyze quantitative expression of cardiomyocyte specific markers, cardiotroponin I and T, connexin 43, GATA4, Mef2c, Nkx2.5 and Tbx5 as determined by real-time PCR (primers listed in table S1). The expression was normalized to that of 18S RNA. The protein expression of sarcomeric actinin was determined by histochemical staining. For EC lineage differentiation, D3-treated and control cells were cultured in medium suitable for inducing endothelial differentiation (10% FBS/EBM-2; Clonetics) medium containing supplements (SingleQuot Kit; Clonetics) for 7 days. mRNA expression for endothelial cell (EC) markers, CD31, Flk1 and VEGFR3 was determined by real-time PCR (primers listed in table 1) and by incubated with DiI-acLDL (Biomedical Technologies) for one hour followed by Isolectin B4 staining. The dual stained cells were identified as endothelial cells.

### Induction of Neuronal and Adipogenic Differentiation

The neuronal differentiation was performed as described by Kusano et al., Nat Med. 11, 1197 (2005), which is hereby incorporated by reference. Briefly, cells were seeded in complete DMEM medium at 5×10⁵ cells per 90-mm sterile culture dish. Suspension cultures were maintained for 24 hours before adding 10 μM all-trans-retinoic acid (Sigma-Aldrich). Cells were cultured for 3 weeks in retinoic acid, and the medium was replaced every 2-3 days. Subsequently, cell aggregates were washed in complete DMEM medium and plated onto poly-L-lysine (10 μg/ml; Sigma-Aldrich)-coated plates in complete DMEM medium containing the mitotic inhibitors fluorodeoxyuridine (10 μM; Sigma-Aldrich), cytosine arabinosine (1 μM; Sigma-Aldrich), and uridine (10 μM; Sigma-Aldrich). The culture dishes were stained for neuronal markers nestin and β-tubulin-III.

The adipogenic differentiation was performed as described by Stewart et al., Stem Cells. 21, 248 (2003). Briefly, the cells were cultured for 21 days in complete DMEM/Ham's F-12 medium containing dexamethasone, insulin and indomethacin. Cells were fixed with 4% paraformaldehyde, washed in 5% isopropanol, and stained for 15 minutes with Oil-Red-O (Sigma Aldrich).

### Immunochemical Staining

For immunochemical staining cells under different culture conditions were cultured on 4 well slides for various times, rinsed once with PBS and fixed with 4% paraformaldehyde (Sigma) in PBS for 30 minutes. The slides were rinsed three times with PBS and then permeabilized with 0.3% of Triton X-100 (Sigma) in PBS for 5 minutes. After 2 washings with PBS, specific primary antibodies diluted in PBS containing 1% FBS were added and incubated overnight at 4° C. After 3 additional washes with PBS, the slides were incubated with the respective secondary antibodies for 1 hour at 37° C. The excess secondary antibodies in the slides were rinsed off by washing in PBS three times. Finally to visualize nuclei, slides were stained with Dapi for 5 minutes and washed 3 times with PBS, allowed to dry for 5 minutes and then mounted on Vectashield mounting medium for fluorescence imaging. The photographs were taken in a Nikon TE200 Digital Imaging system.

### Determination of Oct4 Promoter Methylation and Bisulfite Genomic Sequencing and Chromatin Immunoprecipitation (ChIP).

Genomic DNA prepared from D3, 3T3/D3 and 3T3 cells was amplified for Oct4 promoter and the PCR product was digested with HpyCH4IV restriction enzymes that cleave at methylated CpG sites. The digested products were analyzed on agarose gels. For genomic bisulphate sequencing, genomic DNA from cells was digested with EcoR1 and was used for bisulphite treatment using EZ DNA methylation-Gold kit essentially following manufacturer's instructions. The treated DNA was ethanol-precipitated and resuspended in water and then amplified by PCR using mouse Oct4 primers (table S1). PCR products were digested with HpyCH4IV (New England Biolabs) restriction enzyme. Because only unmethylated cytosine residues were changed to thymines by the sodium bisulphite reaction, PCR fragments from nonmethylated genomic DNA were resistant to HpyCH4IV, and those from methylated DNA were digested by the enzymes. The resultant products of restriction mapping were assessed by agarose gel electrophoresis. The remaining PCR products were purified using a commercially available purification column provided by the Wizard DNA Clean-Up system (Promega, Madison, Wis.). Purified samples were directly sequenced to determine the methylation status of all 10 CpG residues present in the amplified promoter region. Chromatin Immunoprecipitation (ChIP) assays were carried out as described in Kishore et al., J Clin Invest. 115L 1785 (2005). Anti-Acetyl H3, anti-acetyl H4 and anti-dimethyl K9 antibodies were purchased from Upstate Biotech and Santa Cruz.

### Genome-Wide Expression Profiling and Gene Expression Analyses.

Affymetrix mouse genome A2 GeneChips were used for hybridization. Using a poly-dT primer incorporating a T7 promoter, double-stranded cDNA was synthesized from 5 μg total RNA using a double-stranded cDNA synthesis kit (Invitrogen, Carlsbad, Calif.). Double-stranded cDNA was purified with the Affymetrix sample cleanup module (Affymetrix, Santa Clara, Calif.). Biotin-labeled cRNA was generated from the double-stranded cDNA template through in-vitro transcription (IVT) with T7 polymerase, and a nucleotide mix containing biotinylated UTP (3′-Amplification Reagents for IVT Labeling Kit; Affymetrix). The biotinylated cRNA was purified using the Affymetrix sample cleanup module. For each sample, 15 μg of IVT product was digested with fragmentation buffer (Affymetrix, Santa Clara, Calif.) for 35 minutes at 94° C., to an average size of 35 to 200 bases. 10 μg of the fragmented, biotinylated cRNA, along with hybridization controls (Affymetrix), was hybridized to a Mouse 430A 2.0 GeneChip for 16 hours at 45° C. and 60 rpm. Arrays were washed and stained according to the standard Antibody Amplification for Eukaryotic Targets protocol (Affymetrix). The stained arrays were scanned at 532 nm using an Affymetrix GeneChip Scanner 3000.

During analysis and for quality control, GeneChip® arrays were first inspected using a series of quality control steps. Present call rates were consistent across the twelve arrays, ranging from 56% to 63%. The hybridization control BioB was found to be present 100% of the time, and the remaining hybridization controls (BioB, BioC, BioC, Cre) were present 100% of the time. Images of all arrays were examined, and no obvious scratches or spatial variation was observed. A visual inspection of the distribution of raw PM probes values for the twelve arrays showed no outlying arrays. Similarly, digestion curves describing trends in RNA degradation between the 5′ end and the 3′ end of each probeset were generated, and all twelve proved comparable. Probe sets with no Present calls across the twelve arrays as well as Affymetrix control probe sets were excluded from further analyses. Raw intensity values for the remaining 17,213 probe sets were processed first by RMA (Robust Multi-Array Average) using the R package affy (40). Specifically, expression values were computed from raw CEL files by first applying the RMA model of probe-specific correction of PM (perfect match) probes. These corrected probe values were then normalized via quantile normalization, and a median polish was applied to compute one expression measure from all probe values. Resulting RMA expression values were log₂-transformed. (Please see the affy manual at www.bioconductor.org/repository/devel/vignette/affy.pdf for details). Distributions of expression values processed via RMA of all arrays were very similar with no apparent outlying arrays. Pearson correlation coefficients and Spearman rank coefficients were computed on the RMA expression values (log₂-transformed) for each set of biological triplicates. Spearman coefficients ranged from 0.990 to 0.996; Pearson coefficients ranged between 0.991 and 0.997. Differential expression of genes was determined by one-way ANOVA on the RMA expression values of each probe set, using the R package limma (30). A multiple testing adjustment (31) was performed on the resulting statistics to adjust the false discovery rate. Differentially expressed probes with adjusted p-value <0.01 and a fold-change of greater than two (absolute log fold change of greater than 1) were extracted for further inspection. Hierarchical clustering was obtained by using the Pearson correlation coefficient and average agglomeration method, and the heatmaps were generated using z-scored probes, in which z-scores (subtraction of mean and division by standard deviation of normalized values) were computed for each probe across all twelve arrays.

### CFP-Transduction and DiI Labeling of Cells for Transplantation.

For tracking of transplanted cells in cardiac tissues, D3-extract treated control NIH3T3 cells were transduced with a lentivirus-GFP construct. For tracking of transplanted cells in hind limb ischemic tissues, the cells were labeled with DiI before the transplantation.

### Teratoma Formation and Histological Analysis.

D3, 3T3 and 3T3/D3 cells were suspended at 1×10⁷ cell/ml in cytokine-reduced matrigel. SCID mice (5 mice/cell type) were injected with 100 μl of cell suspension (1×10⁶ cells) subcutaneously into dorsal flank. Tumors were resurrected when they reached the size of approximately 3 cm (3 week from injection for D3 cells and 7 weeks following 3T3/D3 cell injections (kinetics shown in FIG. 7B). Half of the dissected tumors were snap frozen, sectioned and were stained with specific primary antibodies.

### Hind Limb Ischemia, Cell Transplantation, and Laser Doppler Imaging.

To ascertain the functional efficacy of reprogrammed D3-extract treated 3T3 cells in a physiologically relevant model of tissue repair, studies were conducted in a well-established mouse hind limb ischemia model. The hind limb ischemia was established by the excision of femoral artery in the left hind limb in 10 male 8-week old mice (Jackson Labs) essentially as described in our prior publication (20). The animals were separated into two groups. Each group received either DiI-labeled-5×10⁴ 3T3 cells or 3T3-D3 cells. The cells were injected at multiple sites into the ischemic muscle. Laser Doppler imaging was carried out to determine blood flow immediately after surgery (day 0) and at day 7 after cell injections. To assess the proliferation of injected cells, a few animals were injected with BrdU intravenously 24 hours before the collection of tissues. Fourteen days after cell transplantation, the tissues were harvested and assayed by histochemical/immuno-fluorescence staining for isolectin B4 (EC identity), Desmin (muscle), BrdU, and DiI followed by fluorescence microscopy. In some experiments, animals were perfused with FITC-BS-1 lectin to identify capillaries before sacrifice and tissue retrieval.

### Mice and Establishment of AMI

All procedures were performed in accordance with the guidelines of the Caritas St. Elizabeth's Institutional Animal Care and Use Committee. The study involved 8-week-old male FVB mice (n=30; Jackson Laboratories). Mice underwent surgery to induce acute myocardial infarction by ligation of the left anterior descending coronary artery, as described before (32, 41). Animals were sub-divided into 3 groups, and received intramyocardial injection of 2.5×10⁴ lentiviral-GFP transduced D3-extract treated cells, 3T3 fibroblast control cells and saline, respectively, in total volume of 10 μl at 5 sites (basal anterior, mid anterior, mid lateral, apical anterior and apical lateral) in the peri-infarct area.

### Physiological Assessments of LV Function Using Echocardiography

Mice underwent echocardiography just before MI (base level) and one, two and four weeks after AMI as described by Iwakura et al., Circulation. 113: 1605 (2006) and Yoon et al., J Clin Invest. 111: 717 (2003). Briefly, transthoracic echocardiography was performed with a 6 to 15 MHz transducer (SONOS 5500, Hewlett Packard). Two-dimensional images were obtained in the parasternal long and short axis and apical 4-chamber views. M-mode images of the left ventricular short axis were taken at just below the level of the mid-papillary muscles. Left ventricular end-diastolic and end-systolic dimensions were measured and functional shorting was determined according to the modified American Society of Echocardiography-recommended guidelines. A mean value of 3 measurements was determined for each sample.

### Histology

Mice were euthanized and the aortas were perfused with saline. The hearts were sliced into 4 transverse sections from apex to base and fixed with 4% paraformaldehyde, methanol or frozen in OCT compound and sectioned into 5-μm thickness. Immunoflurorescence staining was performed to evaluate cardiomyocyte and EC differentiation of transplanted cells; to determine myocardial proliferation (ki67 staining) and apoptosis (TUNEL), essentially as described by Yoon et al., J Clin Invest. 111, 717 (2003).

### Fibrosis Area

For the measurement of fibrosis, tissues sections were frozen in OCT compound and sectioned for elastic tissue/trichrome to measure the average ratio of the external circumference of fibrosis area to LV area.

### Statistical Analyses.

All experiments were carried out at least 3 times with similar results. Results are presented as mean±SEM. Comparisons were done by ANOVA (GB-STAT; Dynamic Microsystems Inc.) or χ² test for percentages. All tests were 2-sided, and a P value of less than 0.05 was considered statistically significant.

### Other Embodiments

From the foregoing description, it will be apparent that variations and modifications may be made to the invention described herein to adopt it to various usages and conditions. Such embodiments are also within the scope of the following claims. The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for generating a reprogrammed cell, the method comprising:
(a) contacting a somatic cell comprising a permeable cell membrane with an embryonic stem cell extract, thereby generating a de-differentiated cell; and
(b) culturing the de-differentiated cell in the presence of at least one agent that induces differentiation, thereby generating a reprogrammed cell.

2. The method of claim 1, wherein the method further comprises providing the cell to a subject for the repair or regeneration of a tissue or organ.

3. (canceled)

4. The method of claim 1, wherein the contacting occurs in an ATP regenerating buffer that comprises one or more of ATP, creatine phosphate, and creatine kinase.

5. The method of claim 1, wherein the de-differentiated cell expresses an embryonic stem cell marker selected from the group consisting of Nanog, SCF, SSEA1, Oct-4, and c-Kit.

6. (canceled)

7. The method of claim 1, wherein the de-differentiated cell has reduced levels of DNA methylation or increased levels of histone acetylation relative to an untreated somatic cell.

8-9. (canceled)

10. The method of claim 1, wherein the agent is selected from the group consisting of LIF, BMP-2, retinoic acid, trans-retinoic acid, dexamethasone, insulin, and indomethacin.

11. The method of claim 1, wherein the cell is cultured under conditions selected from the group consisting of:
in the presence of LIF and BMP-2 to generate a reprogrammed cell that expresses a cardiomyocyte specific gene selected from the group consisting of connexin43, Mef2C, Nkx2.5, GATA4, cardiac troponin I, cardiac troponin T, and Tbx5;
in the presence of fibronectin and 10% fetal bovine serum to generate a reprogrammed cell that expresses an endothelial cell marker that is CD31 or Flk-1;
in the presence of all-trans retinoic acid or a derivative thereof to generate a reprogrammed cell that expresses a neuronal marker selected from the group consisting of nestin and β-tubulin; and
in the presence of at least one of retinoic acid, dexamethasone, insulin, and indomethacin to generate a reprogrammed cell that is positive for Oil red O or acetylated LDL uptake.

12-19. (canceled)

20. A method for repairing or regenerating a tissue in a subject, the method comprising
(a) obtaining the reprogrammed cell of claim 1, and
(b) administering the cell to the subject to repair or regenerate a tissue.

21-25. (canceled)

26. The method of claim 20, wherein the cell is administered directly to a subject at a site where an increase in cell number is desired.

27. (canceled)

30. A method of ameliorating an ischemic condition in a subject, the method comprising
(a) contacting a fibroblast cell comprising a permeable cell membrane with an embryonic stem cell extract in an ATP regenerating buffer;
(b) culturing the cell in the presence of LIF and BMP-2 to generate an endothelial cell; and
(c) administering the endothelial cell of step (b) into a muscle tissue of the subject, thereby ameliorating an ischemic condition.

31. (canceled)

32. The method of claim 30, wherein the method reduces apoptosis, increases cell proliferation, increases function, or increases perfusion of the muscle tissue.

33-43. (canceled)

44. A reprogrammed cell obtained by the method of claim 1.

45. The reprogrammed cell of claim 44, wherein the cell is a differentiated cardiomyocyte, endothelial cell, neuronal cell, adipocyte, or a precursor thereof.

46. The reprogrammed cell of claim 44, wherein the cell expresses a cardiomyocyte marker selected from the group consisting of connexin43, Mef2C, Nkx2.5, GATA4, cardiac troponin I, cardiac troponin T, and Tbx5.

47. The reprogrammed cell of claim 44, wherein the cell is an endothelial cell that expresses an endothelial marker that is CD31 or Flk-1.

48. The reprogrammed cell of claim 44, wherein the cell is a neuronal cell that expresses a neuronal marker that is nestin or β-tubulin.

49. The reprogrammed cell of claim 44, wherein the cell is an adipocyte cell that is positive for Oil red O.

50. A tissue comprising the reprogrammed cell of claim 44.

51. A pharmaceutical composition comprising an effective amount of a cell of claim 44 in a pharmaceutically acceptable excipient for administration to a subject.

52. A kit for tissue repair or regeneration comprising a reprogrammed cell obtained by the method of claim 1 and instructions for use of the cell in methods of tissue repair or regeneration.
